(19) United States
(12) Patent Application Publication
(10) Pub. No.: US 2009/0271164 A1
(43) Pub. Date: Oct. 29, 2009

(54) PREDICTING LONG-TERM EFFICACY OF A COMPOUND IN THE TREATMENT OF PSORIASIS

(76) Inventors: Joanna Z. Peng, New York, NY (US); Peter A. Noertersheuser, Großkarlbach (DE)

Correspondence Address: McCarter & English, LLP / Abbott Laboratories Ltd., 265 Franklin Street, Boston, MA, 02110, US

(21) Appl. No.: 12/346,995

(22) Filed: Dec. 31, 2008

Related U.S. Application Data
(60) Provisional application No. 61/009,906, filed on Jan. 3, 2008.
(60) Provisional application No. 61/128,202, filed on May 20, 2008.

(51) Int. Cl. G06G 7/48; G06F 17/00; G06F 19/00; G01N 33/48

(52) U.S. Cl. 703/11; 707/104.1; 702/19; 707/E17.009

(57) ABSTRACT

The invention provides a method for predicting the efficacy of a compound for treating psoriasis based on a pharmacokinetic/pharmacodynamic model.

### RELATED APPLICATIONS

This application claims the benefit of priority to U.S. provisional patent application No. 61/009,906, filed on Jan. 3, 2008 and U.S. provisional patent application No. 61/128,202, filed May 20, 2008, the contents each of which are hereby incorporated by reference in their entirety

### BACKGROUND OF THE INVENTION

Psoriasis is a chronic, immune-mediated disease affecting 1-3% of the population worldwide (Jacobson and Kimball, Epidemiology: Psoriasis In: Psoriasis and Psoriatic Arthritis (Eds: Gordon K B, Ruderman E M). Springer-Verlag Berlin Heidelberg, Germany; 2005:47-56), with the greatest disease prevalence occurring in North America and Europe (Krueger and Duvic, J. Invest. Dermatol, 102:145-185, 1994). The most common form of psoriasis is plaque-type psoriasis, present in 65-86% of patients and characterized by the presence of thick, scaly plaques. Based on the National Psoriasis Foundation's definitions of moderate to severe psoriasis, the prevalence of moderate to severe psoriasis in the United States is estimated at 0.31% of persons age 18 or older (Stem et al., J. Investig. Dermatol. Symp. Proc. 9:136-139, 2004). Patients with psoriasis report reduction in physical functioning and mental functioning comparable to that observed in patients with cancer, arthritis, hypertension, heart disease, diabetes, and depression (Rapp et al., J. Am. Acad. Dermatol. 41(3Pt1):401-407, 1999). In a US survey of the impact of psoriasis on quality of life, respondents reported difficulties in the workplace, difficulties socializing with family members and friends, exclusion from public facilities, difficulties in getting a job, and contemplation of suicide (Krueger et al., Arch. Dermatol., 137:280-284, 2001).

Traditionally, treatment for psoriasis has included medications that suppress the growth of skin cells. Treatment approaches for psoriasis often include creams and ointments, oral medications, and phototherapy. In recent years, biologic response modifiers that inhibit certain cytokines have become a potential new avenue of treatment for psoriasis patients. For example, tumor necrosis factor (TNF) is a cytokine involved in inflammatory response and scientific evidence suggests it plays a fundamental role in the pathogenesis of psoriasis (Kreuger et al. (2004) Arch Dermatol 140:218; Kupper (2003) N Engl J Med 349:1987).

However, while a number of local and systemic therapies have been reported to be useful for treating psoriasis, there remains a need for determining or predicting the long-term efficacy of such treatments.

### SUMMARY OF THE INVENTION

The present invention is based, at least in part, on the discovery of a pharmacokinetic and pharmacodynamic modeling and simulation approach which was demonstrated to accurately predict the long-term efficacy of a compound for treating psoriasis.

Accordingly, in one aspect, the present invention features a method for predicting the efficacy of a compound, for the treatment of psoriasis using a pharmacokinetic/pharmacodynamic model. The methods of the invention include, in one embodiment, creating a pharmacokinetic model describing the pharmacokinetic profile of the compound and a pharmacodynamic model to predict the long term efficacy of the compound based on the calculation of an indices for psoriasis e.g., PASI, PGA, DLQI, status. In a preferred embodiment, the pharmacodynamic model is used to calculate the PASI score. In another embodiment, the methods of the invention may be used for predicting the plateau PASI response rate of a psoriasis therapy. In a preferred embodiment, the plateau PASI 75 response rate for a psoriasis therapy is predicted.

In one preferred embodiment, the pharmacokinetic model contains a central compartment, the central compartment describing a concentration of the compound at a given time. In one embodiment, the pharmacodynamic model used in the methods of the invention an indirect response. In one embodiment, the pharmacodynamic model is a two-step indirect response model with an Eₘₐₓ concentration-response relationship. In a preferred embodiment, the pharmacodynamic model is a two-step indirect model with a linear concentration-response relationship.

In a one embodiment, the method of the present invention also includes calculating the inter-individual errors for the rate into the second step of the pharmacodynamic model and the rate out of the second step of the pharmacodynamic model and/or creating a residual error model combining additive and proportional error as a weighting factor. In another embodiment, the pharmacodynamic model used in the methods of the invention includes exponential inter-individual error terms (e.g., Kᵢₙ and K₄₀).

In certain embodiments of the methods of the invention, the treatment for psoriasis assessed according to the methods of the invention is a systemic treatment. In one embodiment, the systemic treatment comprises a TNFα inhibitor. In another embodiment, the systemic treatment comprises a corticosteroid. In one embodiment, the treatment comprises methotrexate. In still another embodiment, the long-term efficacy of a combination of compounds is predicted using the methods of the invention.

In certain embodiments, the methods of the invention are used to predict the efficacy two or more psoriasis treatments. In other embodiments the methods of the invention are used to predict the efficacy of two or more dosage regimens of a psoriasis treatment.

In certain embodiments, the methods of the invention are used to predict the efficacy of one or more psoriasis treatments and/or dosage regimens in a patient population containing subjects diagnosed with psoriasis. In one embodiment, the psoriasis is moderate to severe (e.g., >10% body surface area involvement and a PASI score of >10). In other embodiments, the patient population is a subpopulation having a common physical characteristic (e.g., age, gender, ethnicity, weight). In another embodiment, the patient population contains subjects who have had a subtherapeutic response to a therapy, who has failed to respond to a therapy, or has lost responsiveness to a previous psoriasis therapy.

In further embodiments, the methods of the invention are used to predict the efficacy one or more psoriasis treatments and/or dosage regimens in an individual. For example, the efficacy of a particular psoriasis treatment or dosage regimen may be predicted using a pharmacokinetic/pharmacodynamic model based on population data from similar patients.

The invention also features computer programs, computer readable media and computer systems which may be used in the methods described herein for predicting the efficacy of a psoriasis treatment for a population or an individual.

Additional embodiments of the invention are provided in the Detailed Description and Examples set forth herein.

### BRIEF DESCRIPTION OF THE DRAWINGS

FIG. 1 illustrates the design schematic of a 16-week multicenter, double-blind, double-dummy study for the evaluation of adalimumab vs. methotrexate vs. placebo.

FIG. 2A is a graph depicting individual predicted PASI scores (IPRED) vs. observed PASI scores.

FIG. 2B is a graph depicting weighted residuals (WRES) vs. time.

FIG. 3A-3C depict graphs of individual PASI scores vs. time profiles (observed and predicted values), along with methotrexate doses. Observed data is represented by black dots; predicted PASI scores are represented by black lines; and methotrexate doses are indicated by vertical lines (needles).

FIG. 4A is a graph depicting the observed and predicted PASI75 response rate over time for a 16 week period. Actual PASI75 response rates are represented by black dotes with error bars indicating 90% CI for the actual PASI75 response rates based on the normal approximation to the binomial distribution. The predicted mean is indicated by the solid black line and the predicted 5^{th} and 95^{th} percentiles are indicated by black dash lines (the area between the 5^{th} and 95^{th} percentiles represents the 90% CI).

FIG. 4B is a graph depicting the observed and predicted PASI75 response rate over time for a 52 week period. Actual PASI75 response rates are represented by black dotes with error bars indicating 90% CI for the actual PASI75 response rates based on the normal approximation to the binomial distribution. The predicted mean is indicated by the solid black line and the predicted 5^{th} and 95^{th} percentiles are indicated by black dash lines (the area between the 5^{th} and 95^{th} percentiles represents the 90% CI).

FIG. 5 illustrates the design schematic of a study to compare the predicted the long-term efficacy of methotrexate with observed adalimumab efficacy data.

FIG. 6 illustrates the two-step indirect exposure-efficacy response model.

FIG. 7 is a bar graph depicting the methotrexate dosage distribution over time.

FIG. 8 is a graph depicting the percentage of patients achieving a PASI75 response rate over time.

### DETAILED DESCRIPTION OF THE INVENTION

### I. Definitions

The terms “psoriasis treatment” or “psoriasis therapy”, used interchangeably herein, refer to one or more agents (also referred to as substances or compounds) that act to interrupt the cycle that causes an increased production of skin cells, thereby reducing inflammation and plaque formation. Psoriasis treatments include topical treatments, light therapy, and systemic medications and combinations thereof. For example, topical psoriasis treatments include, but are not limited to, corticosteroids, vitamin D analogues, anthralin, retinoids, calcineurin inhibitors, coal tar and moisturizers. Light therapy (phototherapy) psoriasis treatments include, but are not limited to UVB phototherapy, narrowband UVB therapy, psoralen plus ultraviolet A (PUVA) and Excimer laser. Systemic psoriasis treatments include, but are not limited to retinoids, methotrexate, azathioprine, cyclosporine, hydroxyurea, and biologics (e.g., TNFα inhibitors), and combinations thereof.

The term “human TNFα” (abbreviated herein as h TNFα or simply hTNF), as used herein, is intended to refer to a human cytokine that exists as a 17 kD secreted form and a 26 kD membrane associated form, the biologically active form of which is composed of a trimer of noncovalently bound 17 kD molecules. The structure of h TNFα is described further in, for example, Pennica, D., et al. (1984) Nature 312:724-729; Davis, J. M., et al. (1987) Biochemistry 26:1322-1326; and Jones, E. Y., et al. (1989) Nature 338:225-228. The term human TNFα is intended to include recombinant human TNFα (rhTNFα), which can be prepared by standard recombinant expression methods or purchased commercially (R & D Systems, Catalog No. 210-TA, Minneapolis, Minn.). TNFα is also referred to as TNF.

The term “TNFα inhibitor” includes agents which interfere with TNFα activity. The term also includes each of the anti-TNFα human antibodies and antibody portions described herein as well as those described in U.S. Pat. Nos. 6,090,382; 6,258,562; 6,509,015, and in U.S. patent application Ser. Nos. 09/801,185 and 10/302,356. In one embodiment, the TNFα inhibitor used in the invention is an anti-TNFα antibody, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA®® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein.

Other examples of TNFα inhibitors include TNF fusion proteins, e.g., etanercept (Enbrel®, Amgen; described in WO 91/03553 and WO 09/406,476), soluble TNF receptor Type I, a pegylated soluble TNF receptor Type I (GEGs TNF-R1), p55TNFR1gG (Lenercept), and recombinant TNF binding proteins, e.g., r-TBP-I, (Serono).

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antigen-binding portion” or “antigen-binding fragment” of an antibody (or simply “antibody portion”), as used herein, refers to one or more fragments of an antibody that retain the ability to specifically bind to an antigen (e.g., hTNFα). It has been shown that the antigen-binding function of an antibody can be performed by fragments of a full-length antibody. Binding fragments include Fab, Fab′, F(ab′)₂, Fabc, Fv, single chains, and single-chain antibodies. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. USA 90:6444-6448; Poljak et al. (1994) Structure 2:1121-1123). Examples, of antibody portions which may be used in the methods of the invention are described in further detail in U.S. Pat. Nos. 6,090,382, 6,258,562, 6,509,015, each of which is incorporated herein by reference in its entirety.

Still further, an antibody or antigen-binding portion thereof may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Chimeric antibodies” refers to antibodies wherein one portion of each of the amino acid sequences of heavy and light chains is homologous to corresponding sequences in antibodies derived from a particular species or belonging to a particular class, while the remaining segment of the chains is homologous to corresponding sequences from another species. In one embodiment, a chimeric antibody or antigen-binding fragment, refers to an antibody in which the variable regions of both light and heavy chains mimics the variable regions of antibodies derived from one species of mammals, while the constant portions are homologous to the sequences in antibodies derived from another species. In another embodiment of the invention, chimeric antibodies are made by grafting CDRs from a mouse antibody onto the framework regions of a human antibody.

“Humanized antibodies” refer to antibodies which comprise at least one chain comprising variable region framework residues substantially from a human antibody chain (referred to as the acceptor immunoglobulin or antibody) and at least one complementarity determining region (CDR) substantially from a non-human-antibody (e.g., mouse). In addition to the grafting of the CDRs, humanized antibodies typically undergo further alterations in order to improve affinity and/or immunogenicity.

The term “multivalent antibody” refers to an antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” or “dual specific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

An “isolated antibody”, as used herein, is intended to refer to an antibody that is substantially free of other antibodies having different antigenic specificities (e.g., an isolated antibody that specifically binds hTNFα is substantially free of antibodies that specifically bind antigens other than hTNFα). An isolated antibody that specifically binds hTNFα may, however, have cross-reactivity to other antigens, such as TNFα molecules from other species. Moreover, an isolated antibody may be substantially free of other cellular material and/or chemicals.

A “neutralizing antibody”, as used herein (or an “antibody that neutralized hTNFα activity”), is intended to refer to an antibody whose binding to hTNFα results in inhibition of the biological activity of hTNFα. This inhibition of the biological activity of hTNFα can be assessed by measuring one or more indicators of hTNFα biological activity, such as hTNFα-induced cytotoxicity (either in vitro or in vivo), hTNFα-induced cellular activation and hTNFα binding to hTNFα receptors. These indicators of hTNFα biological activity can be assessed by one or more of several standard in vitro or in vivo assays known in the art (see U.S. Pat. No. 6,090,382). Preferably, the ability of an antibody to neutralize hTNFα activity is assessed by inhibition of hTNFα-induced cytotoxicity of L929 cells. As an additional or alternative parameter of hTNFα activity, the ability of an antibody to inhibit hTNFα-induced expression of ELAM-1 on HUVEC, as a measure of hTNFα-induced cellular activation, can be assessed.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

The term “IC₅₀” as used herein, is intended to refer to the concentration of a substance required to inhibit the biological endpoint of interest, e.g., reduce inflammation, plaque formation, neutralize cytotoxicity activity.

The term “dose,” as used herein, refers to an amount of a substance which is administered to a subject.

The term “dosing”, as used herein, refers to the administration of a substance (e.g., an anti-TNFα antibody) to achieve a therapeutic objective (e.g., treatment of psoriasis).

A “dosing regimen” describes a treatment schedule for a substance, e.g., a treatment schedule over a prolonged period of time and/or throughout the course of treatment, e.g. administering a first dose of a substance at week 0 followed by a second dose of a substance on a daily, twice weekly, thrice weekly, weekly, biweekly or monthly dosing regimen.

The terms “biweekly dosing regimen”, “biweekly dosing”, and “biweekly administration”, as used herein, refer to the time course of administering a substance (e.g., an anti-TNFα antibody) to a subject to achieve a therapeutic objective, e.g, throughout the course of treatment. The biweekly dosing regimen is not intended to include a weekly dosing regimen. Preferably, the substance is administered every 9-19 days, more preferably, every 11-17 days, even more preferably, every 13-15 days, and most preferably, every 14 days. In one embodiment, the biweekly dosing regimen is initiated in a subject at week 0 of treatment. In another embodiment, a maintenance dose is administered on a biweekly dosing regimen. In one embodiment, both the loading and maintenance doses are administered according to a biweekly dosing regimen. In one embodiment, biweekly dosing includes a dosing regimen wherein doses of a substance are administered to a subject every other week beginning at week 0. In one embodiment, biweekly dosing includes a dosing regimen where doses of a substance are administered to a subject every other week consecutively for a given time period, e.g., 4 weeks, 8 weeks, 16, weeks, 24 weeks, 26 weeks, 32 weeks, 36 weeks, 42 weeks, 48 weeks, 52 weeks, 56 weeks, etc. Biweekly dosing methods are also described in US 20030235585, incorporated by reference herein.

The term “multiple-variable dose” includes different doses of a substance which are administered to a subject for therapeutic treatment. “Multiple-variable dose regimen” or “multiple-variable dose therapy” describes a treatment schedule which is based on administering different amounts of a substance at various time points throughout the course of treatment. Multiple-variable dose regimens are described in PCT application no. PCT/US05/12007 and US 20060009385, which is incorporated by reference herein.

The term “maintenance therapy” or “maintenance dosing regime” refers to a treatment schedule for a subject or patient diagnosed with a disorder/disease, e.g., psoriasis, to enable them to maintain their health in a given state, e.g, remission. Generally, the first goal of treatment of psoriasis is to induce remission in the subject in need thereof. The next challenge is to keep the subject in remission. Maintenance doses may be used in a maintenance therapy for maintaining remission in a subject who has achieved remission of a disease or who has reached a state of the disease which is advantageous, e.g. reduction in symptoms. In one embodiment, a maintenance therapy of the invention is used for a subject or patient diagnosed with a disorder/disease, e.g., psoriasis to enable them to maintain their health in a state which is completely free of symptoms associated with the disease. In one embodiment, a maintenance therapy of the invention is used for a subject or patient diagnosed with a disorder/disease, e.g., psoriasis, to enable them to maintain their health in a state which is substantially free of symptoms associated with the disease. In one embodiment, a maintenance therapy of the invention is used for a subject or patient diagnosed with a disorder/disease, e.g., psoriasis, to enable them to maintain their health in a state where there is a significant reduction in symptoms associated with the disease.

The term “induction dose” or “loading dose,” used interchangeably herein, refers to the first dose of a substance which is initially used to induce remission of psoriasis. Often, the loading dose is larger in comparison to the subsequent maintenance or treatment dose. The induction dose can be a single dose or, alternatively, a set of doses.

In one embodiment, an induction dose is subsequently followed by administration of smaller doses of the substance, e.g., the treatment or maintenance dose. The induction dose is administered during the induction or loading phase of therapy. In one embodiment of the invention, the induction dose is at least twice the given amount of the treatment dose.

The term “treatment phase” or “maintenance phase”, as used herein, refers to a period of treatment comprising administration of a substance to a subject in order to maintain a desired therapeutic effect, i.e., maintaining remission of psoriasis.

The term “maintenance dose” or “treatment dose” is the amount of a substance taken by a subject to maintain or continue a desired therapeutic effect. A maintenance dose can be a single dose or, alternatively, a set of doses. A maintenance dose is administered during the treatment or maintenance phase of therapy. In one embodiment, a maintenance dose(s) is smaller than the induction dose(s) and can be equal to each other when administered in succession.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of predicting the efficacy of psoriasis therapies comprising combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “treatment,” as used within the context of the present invention, is meant to include therapeutic treatment, as well as prophylactic or suppressive measures, for the treatment of psoriasis. For example, the term treatment may include administration of a substance prior to or following the onset of psoriasis thereby preventing or removing signs of the disease or disorder. As another example, administration of a substance after clinical manifestation of psoriasis to combat the symptoms and/or complications and disorders associated with psoriasis comprises “treatment” of the disease. Further, administration of the agent after onset and after clinical symptoms and/or complications have developed where administration affects clinical parameters of the disease or disorder and perhaps amelioration of the disease, comprises “treatment” of the psoriasis. In one embodiment, treatment of psoriasis in a subject comprises inducing and maintaining remission of psoriasis in a subject. In another embodiment, treatment of psoriasis in a subject comprises maintaining remission of psoriasis in a subject.

Those “in need of treatment” include mammals, such as humans, already having psoriasis, including those in which the disease or disorder is to be prevented, and individuals who have psoriasis but have failed to respond or have lost responsiveness to other psoriasis treatments.

The term “efficacy” as used herein refers to the extent to which a treatment produces a beneficial result, e.g., and improvement in one or more symptoms of the disease. For example, the efficacy of a psoriasis treatment may be predicted using standard therapeutic indices for psoriasis including, but not limited to, PASI, DLQI, PGA and the like. “Long-term efficacy” refers to the ability of a treatment to maintain a beneficial result over a period of time, e.g., at least about 16 weeks, 26 weeks, 32 weeks, 36 weeks, 40 weeks, 48 weeks, 52 weeks or longer.

The term “pharmacokinetics” refers to the study of the time course of drug and metabolite levels in different fluids, tissues, and excreta of the body and the mathematical relationships required to interpret the related data.

The term “pharmacodynamics” refers to the study of the action of a drug in the body over a period of time including the processes of absorption, distribution, localization in the tissues, biotransformation, and excretion.

The term “absorption” refers to the transfer of a substance across a physiological barrier as a function of time and initial concentration. The amount or concentration of the compound on the external and/or internal side of the barrier is a function of transfer rate and extent, and may range from zero to unity.

The term “bioavailability” refers to the fraction of an administered dose of a substance that reaches the sampling site and/or site of action. This value may range from zero to unity and can be assessed as a function of time.

A “Computer Readable Medium” refers to a medium for temporary or permanent storing, retrieving and/or manipulating information using a computer including, but not limited to, optical, digital, magnetic mediums and the like (e.g., computer diskette, CD-ROMs, computer hard drive), as well as remote access mediums such as internet or intranet systems.

An “Input/Output System” is an interface between the user and a computer system.

Various aspects of the invention are described in further detail herein.

### II. Psoriasis

Psoriasis is described as a skin inflammation (irritation and redness) characterized by frequent episodes of redness, itching, and thick, dry, silvery scales on the skin. In particular, lesions are formed which involve primary and secondary alterations in epidermal proliferation, inflammatory responses of the skin, and an expression of regulatory molecules such as lymphokines and inflammatory factors. Psoriatic skin is morphologically characterized by an increased turnover of epidermal cells, thickened epidermis, abnormal keratinization, inflammatory cell infiltrates into the epidermis and polymorphonuclear leukocyte and lymphocyte infiltration into the epidermis layer resulting in an increase in the basal cell cycle. Psoriasis often involves the nails, which frequently exhibit pitting, separation of the nail, thickening, and discoloration. Psoriasis is often associated with other inflammatory disorders, for example arthritis, including rheumatoid arthritis, inflammatory bowel disease (IBD), and Crohn's disease.

Evidence of psoriasis is most commonly seen on the trunk, elbows, knees, scalp, skin folds, or fingernails, but it may affect any or all parts of the skin. Normally, it takes about a month for new skin cells to move up from the lower layers to the surface. In psoriasis, this process takes only a few days, resulting in a build-up of dead skin cells and formation of thick scales. Symptoms of psoriasis include: skin patches, that are dry or red, covered with silvery scales, raised patches of skin, accompanied by red borders, that may crack and become painful, and that are usually lovated on the elbows, knees, trunk, scalp, and hands; skin lesions, including pustules, cracking of the skin, and skin redness; joint pain or aching which may be associated with of arthritis, e.g., psoriatic arthritis.

The diagnosis of psoriasis is usually based on the appearance of the skin. Additionally a skin biopsy, or scraping and culture of skin patches may be needed to rule out other skin disorders. An x-ray may be used to check for psoriatic arthritis if joint pain is present and persistent.

In one embodiment of the invention, the long term efficacy of a therapy used to treat psoriasis, including chronic plaque psoriasis, guttate psoriasis, inverse psoriasis, pustular psoriasis, pemphigus vulgaris, erythrodermic psoriasis, psoriasis associated with inflammatory bowel disease (IBD), and psoriasis associated with rheumatoid arthritis (RA) is determined. Specific types of psoriasis included in the treatment methods of the invention are described in detail below:

a. Chronic Plaque Psoriasis

Chronic plaque psoriasis (also referred to as psoriasis vulgaris) is the most common form of psoriasis. Chronic plaque psoriasis is characterized by raised reddened patches of skin, ranging from coin-sized to much larger. In chronic plaque psoriasis, the plaques may be single or multiple, they may vary in size from a few millimeters to several centimeters. The plaques are usually red with a scaly surface, and reflect light when gently scratched, creating a “silvery” effect. Lesions (which are often symmetrical) from chronic plaque psoriasis occur all over body, but with predilection for extensor surfaces, including the knees, elbows, lumbosacral regions, scalp, and nails. Occasionally chronic plaque psoriasis can occur on the penis, vulva and flexures, but scaling is usually absent. Diagnosis of patients with chronic plaque psoriasis is usually based on the clinical features described above. In particular, the distribution, color and typical silvery scaling of the lesion in chronic plaque psoriasis are characteristic of chronic plaque psoriasis.

b. Guttate Psoriasis

Guttate psoriasis refers to a form of psoriasis with characteristic water drop shaped scaly plaques. Hares of guttate psoriasis generally follow an infection, most notably a streptococcal throat infection. Diagnosis of guttate psoriasis is usually based on the appearance of the skin, and the fact that there is often a history of recent sore throat.

c. Inverse Psoriasis

Inverse psoriasis is a form of psoriasis in which the patient has smooth, usually moist areas of skin that are red and inflammed, which is unlike the scaling associated with plaque psoriasis. Inverse psoriasis is also referred to as intertiginous psoriasis or flexural psoriasis. Inverse psoriasis occurs mostly in the armpits, groin, under the breasts and in other skin folds around the genitals and buttocks, and, as a result of the locations of presentation, rubbing and sweating can irriate the affected areas.

d. Pustular Psoriasis

Pustular psoriasis is a form of psoriasis that causes pus-filled blisters that vary in size and location, but often occur on the hands and feet. The blisters may be localized, or spread over large areas of the body. Pustular psoriasis can be both tender and painful, can cause fevers.

e. Other Psoriasis Disorders

Other examples of psoriatic disorders which can be treated with the TNFα antibody of the invention include erythrodermic psoriasis, vulgaris, psoriasis associated with IBD, and psoriasis associated with arthritis, including rheumatoid arthritis.

### Clinical Severity of Psoriasis

Severity of psoriasis may be determined according to standard clinical definitions. For example, the Psoriasis Area and Severity Index (PASI) is used by dermatologists to assess psoriasis disease intensity. This index is based on the quantitative assessment of three typical signs of psoriatic lesions: erythema, infiltration, and desquamation, combined with the skin surface area involvement in the four main body areas (head, trunk, upper extremities and lower extremities). Since its development in 1978, this instrument has been used throughout the world by clinical investigators (Fredriksson T, Petersson U: Severe psoriasis—oral therapy with a new retinoid. Dermatologica 1978; 157: 238-41.) PASI scores range from 0-72, with higher scores indicating greater disease severity. Improvements in psoriasis are indicated as PASI 50 (a 50 percent improvement in PASI from baseline), PASI 75 (a 75 percent improvement in PASI from baseline), PASI 90 (a 90 percent improvement in PASI from baseline), and PASI 100 (a 100 percent improvement in PASI from baseline).

The Physicians Global Assessment (PGA) is used to assess psoriasis activity and follow clinical response to treatment. It is a six-point score that summarizes the overall quality (erythema, scaling and thickness) and extent of plaques relative to the baseline assessment. A patient's response is rated as worse, poor (0-24%), fair (25-49%), good (50-74%), excellent (75-99%), or cleared (100%) (van der Kerkhof P. The psoriasis area and severity index and alternative approaches for the assessment of severity: persisting areas of confusion. Br J Dermatol 1997; 137:661-662).

Other measures of improvements in the disease state of a subject having psoriasis include clinical responses, such as the Dermatology Life Quality Index (DLQI). Characteristics of the DLQI include:
ten items on an overall scoring range of 0-30; higher scores represent greater quality of life impairment and lower scores represent lower quality of life impairment;
well-established properties of reliability and validity for the DLQI total score in a dermatology setting (see Badia et al. (1999) Br J Dermatol 141:698; Finlay et al. (1994) Clin Exp Dermatol 19:210; and Shikier et al. (2003) Health and Quality of Life Outcomes 1:53; Feldman et al. (2004)) Br J Dermatol 150:317; Finlay et al. (2003) Dermatology 206:307; Gordon et al. (2003) JAMA 290:3073; Gottlieb et al. (2003) Arch Dermatol 139:1627; Leonardi et al. (2003) N Engl J Med 349:2014; and Menter et al. (2004) J Drugs Dermatol 3:27));
six subcategories: symptoms and feelings; daily activities; leisure; work/school;
personal relationships; and treatment;
all data are observed values. Patients who discontinued before the time point were not included in this analysis.
   Ranges of DLQI scores can be evaluated for their correspondence to categories of disease impact.

The Short Form 36 Health Survey (SF-36) is a 36-item general health status instrument often used in clinical trials and health services research. It consists of eight domains: Physical Function, Role Limitations-Physical, Vitality, General Health Perceptions, Bodily Pain, Social Function, Role Limitations—Emotional, and Mental Health. Two overall summary scores can be obtained—a Physical Component Summary (PCS) score and a Mental Component Summary (MCS) score. The PCS and MCS scores range from 0-100, with higher scores indicating better health. The SF-36 has been used in a wide variety of studies involving psoriasis, including descriptive studies and clinical research studies, and has demonstrated good reliability and validity. Internal consistency for most SF-36 domains is greater than 0.70. The SF-36 has been shown to discriminate between known groups in a variety of diseases, is reproducible, and is responsive to longitudinal clinical changes.

The EQ-5D is a six-item, preference-based instrument designed to measure general health status. The EQ-5D has two sections: The first consists of five items to assess degree of physical functioning (mobility, self-care, usual activities, pain/discomfort, and anxiety/depression). Items are rated on a three-point scale ranging from “No Problem” to “Extreme Problem” or “Unable to Do.” Each pattern of scores for the five items is linked to an index score that has a value ranging from 0-1, indicating the health utility of that person's health status. The specific linkage can differ from country to country, reflecting differences in cultures to the item responses. The second section is the sixth item on the EQ-5D, which is a visual analog scale with endpoints of “100” or “Best Imaginable Health,” and “0” or “Worst Imaginable Health.” It offers a simple method for the respondents to indicate how good or bad their health statuses are “today.” The score is taken directly from the patients' responses.

### II. Psoriasis Treatments

The long-term efficacy of substances for treating psoriasis may be assessed according to the methods of the invention. In preferred embodiments, the long-term efficacy of a systemic treatment for psoriasis is predicted according to the methods of the invention. In one embodiment, the substance is an oral medication, e.g., methotrexate. In another embodiment, the substance is administered parenterally, e.g., a TNFα inhibitor. In still another embodiment, the long-term efficacy of a combination treatment is predicted. In another embodiment, the long-term efficacy of a dosing regimen for a psoriasis treatment is predicted. In another embodiment, the long-term efficacy of a pharmaceutical formulation containing a substance for the treatment of psoriasis is predicted. In other embodiments, the long-term efficacy of two or more different psoriasis treatments, different dosing regimens, different pharmaceutical formulations, etc., are compared.

It should further be understood that the agents set forth below are illustrative for purposes and not intended to be limited.

a. Topical Treatments

Topical corticosteroids are powerful anti-inflammatory drugs are the most frequently prescribed medications for treating mild to moderate psoriasis. They slow cell turnover by suppressing the immune system, which reduces inflammation and relieves associated itching. Topical corticosteroids range in strength, from mild to very strong. Low-potency corticosteroid ointments are usually recommended for sensitive areas such as the face and for treating widespread patches of damaged skin. Stronger corticosteroid ointment for small areas of the skin, for stubborn plaques on the hands or feet, or when other treatments fail. (http://www.psoriasis.org/treatment/psoriasis/steroids/potency.php) Vitamin D analogues are synthetic forms of vitamin D reduce skin inflammation and help prevent skin cells from reproducing. For example, Calcipotriene (Dovonex) is a prescription cream, ointment or solution containing a vitamin D analogue that may be used alone to treat mild to moderate psoriasis or in combination with other topical medications or phototherapy.

Anthralin is a medication believed to normalize DNA activity in skin cells and to reduce inflammation. Anthralin (e.g., Dritho-Scalp or Psoriatec) can remove scale and smooth skin, but it stains virtually anything it touches, including skin, clothing, countertops and bedding. Anthralin is sometimes used in combination with ultraviolet light.

Topical retinoids are commonly used to treat acne and sun-damaged skin, but tazarotene (Tazorac) was developed specifically for the treatment of psoriasis. Like other vitamin A derivatives, it normalizes DNA activity in skin cells. The most common side effect is skin irritation.

Calcineurin inhibitors (e.g., tacrolimus and pimecrolimus) are only approved for the treatment of atopic dermatitis, but studies have shown them to be effective at times in the treatment of psoriasis as well. Calcineurin inhibitors are thought to disrupt the activation of T cells, which in turn reduces inflammation and plaque buildup.

Coal tar, which is a thick, black byproduct of the manufacture of gas and coke, coal tar is probably the oldest treatment for psoriasis. It reduces scaling, itching and inflammation.

b. Phototherapy

When exposed to UV rays in sunlight or artificial light, the activated T cells in the skin die. This slows skin cell turnover and reduces scaling and inflammation. UVB phototherapy from an artificial light source may improve mild to moderate psoriasis symptoms. UVB phototherapy, also called broadband UVB, can be used to treat single patches, widespread psoriasis and psoriasis that resists topical treatments.

Narrowband UVB therapy is usually administered two or three times a week until the skin improves, then maintenance may require only weekly sessions. Narrowband UVB therapy may cause more severe and longer-lasting burns, however.

Photochemotherapy, or psoralen plus ultraviolet A (PUVA) involves taking a light-sensitizing medication (psoralen) before exposure to UVA light. UVA light penetrates deeper into the skin than does UVB light, and psoralen makes the skin more sensitive to the effects of UVA exposure. This more aggressive treatment consistently improves skin and is often used for more severe cases of psoriasis. PUVA involves two or three treatments a week for a prescribed number of weeks.

Excimer laser is a form of light therapy, used for mild to moderate psoriasis, treats only the involved skin. A controlled beam of UVB light is aimed at the psoriasis plaques to control scaling and inflammation. Healthy skin surrounding the patches remains undamaged. Excimer laser therapy requires fewer sessions than does traditional phototherapy because more powerful UVB light is used.

Pulsed dye lasers are approved for treating chronic, localized plaque lesions. Pulsed dye lasers emit a different form of light than UVB units and the excimer laser and destroy the tiny blood vessels that contribute to and support the formation of psoriasis lesions.

Combining UV light with other treatments such as retinoids frequently improves phototherapy's effectiveness. Combination therapies are often used after other phototherapy options are ineffective. Some doctors give UVB treatment in conjunction with coal tar, called the Goeckerman treatment. The two therapies together are more effective than either alone because coal tar makes skin more receptive to UVB light. Another method, the Ingram regimen, combines UVB therapy with a coal tar bath and an anthralin-salicylic acid paste that's left on the skin for several hours or overnight.

c. Oral Medications

Retinoids, which are related to vitamin A, are group of drugs that may reduce the production of skin cells in people with severe psoriasis who don't respond to other therapies.

Methotrexate helps psoriasis by decreasing the production of skin cells, suppressing inflammation and reducing the release of histamine, a substance involved in allergic reactions. It may also slow the progression of arthritis in some people with psoriatic arthritis. Methotrexate is generally well tolerated in low doses, but when used for long periods it can cause a number of serious side effects, including severe liver damage and decreased production of red and white blood cells and platelets. Taking 1 milligram of folic acid on a daily basis may help reduce some of the common side effects associated with methotrexate.

Azathioprine is a potent anti-inflammatory drug that may be used to treat severe psoriasis when other treatment options fail. Taken long term, azathioprine increases the risk of developing cancerous or noncancerous growths (neoplasias) and certain blood disorders. Other potential side effects include nausea and vomiting, bruising more easily than normal, and fatigue.

Cyclosporine works by suppressing the immune system and is thought to be similar to methotrexate in effectiveness. Like other immunosuppressant drugs, cyclosporine increases the risk of infection and other health problems, including cancer.

Other systemic drugs in include Accutane, Hydrea, mycophenolate mofetil, sulfasalazine, 6-Thioguanine. Hydroxyurea may be used with phototherapy treatments.

d. TNFα Inhibitors

TNFα inhibitors include TNFα antibodies, or an antigen-binding fragment thereof, including chimeric, humanized, human antibodies, dual specific antibodies and single chain antibodies. Examples of TNFα antibodies which may be used in the invention include, but not limited to, infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, 6,090,382, 6,258,562, and 6,509,015, each of which is incorporated by reference herein.

Chimeric, humanized, human, and dual specific antibodies for use in the methods of the invention can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539. To create a scFv gene, the VH- and VL-encoding DNA fragments are operatively linked to another fragment encoding a flexible linker, e.g., encoding the amino acid sequence (Gly4-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

An antibody or antibody portion used in the methods of the invention is also intended to include derivatized and otherwise modified forms of the human anti-hTNFα antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag). In another example, the constant region of the antibody is modified to reduce at least one constant region-mediated biological effector function relative to an unmodified antibody (see e.g., Canfield, S. M. and S. L. Morrison (1991) J. Exp. Med. 173:1483-1491; and Lund, J. et al. (1991) J. of Immunol. 147:2657-2662). In another example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety).

Other examples of TNFα inhibitors which may be used in the methods of the invention include etanercept (Enbrel, described in WO 91/03553 and WO 09/406,476), soluble TNF receptor Type I, a pegylated soluble TNF receptor Type I (PEGs TNF-R1), p55TNFR1gG (Lenercept), and recombinant TNF binding protein (r-TBP-I) (Serono).

e. Combination Therapies

The long-term efficacy of psoriasis treatments may be predicted according to the methods of the invention either alone or in combination with an additional therapeutic agent. In certain embodiments, the additional agent can be a therapeutic agent art-recognized as being useful to treat psoriasis. In other embodiments, the additional agent also can be an agent that imparts a beneficial attribute to the therapeutic composition, e.g., an agent which affects the viscosity of the composition.

It should further be understood that the combinations which are to be included within this invention are those combinations useful for their intended purpose. The agents set forth below are illustrative for purposes and not intended to be limited. The combinations, which are part of this invention, can be a substance for treating psoriasis and at least one additional agent selected from the lists below. The combination can also include more than one additional agent, e.g., two or three additional agents if the combination is such that the formed composition can perform its intended function.

For example, in certain embodiments, the psoriasis treatments described herein may be used in combination with additional therapeutic agents such as a Disease Modifying Anti-Rheumatic Drug (DMARD) or a Nonsteroidal Antiinflammatory Drug (NSAID) or a steroid or any combination thereof. Preferred examples of a DMARD are hydroxychloroquine, leflunomide, methotrexate, parenteral gold, oral gold and sulfasalazine. Preferred examples of non-steroidal anti-inflammatory drug(s) also referred to as NSAIDS include drugs like ibuprofen. Other preferred combinations are corticosteroids including prednisolone; the well known side effects of steroid use can be reduced or even eliminated by tapering the steroid dose required when treating patients in combination with other psoriasis treatments.

Preferred agents for use in combinations of therapeutic agents may interfere at different points in the autoimmune and subsequent inflammatory cascade; preferred examples include TNF antagonists such as soluble p55 or p75 TNF receptors, derivatives, thereof, (p75TNFR1gG (Enbrel™) or p55TNFR1gG (Lenercept), chimeric, humanized or human TNF antibodies, or a fragment thereof, including infliximab (Remicade®, Johnson and Johnson; described in U.S. Pat. No. 5,656,272, incorporated by reference herein), PSORIASIS P571 (a humanized monoclonal anti-TNF-alpha IgG4 antibody), PSORIASIS P 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA®® Abbott Laboratories, a human anti-TNF mAb, described in U.S. Pat. No. 6,090,382 as D2E7). Additional TNF antibodies which can be used in the invention are described in U.S. Pat. Nos. 6,593,458; 6,498,237; 6,451,983; and 6,448,380, each of which is incorporated by reference herein. Other combinations including TNFα converting enzyme (TACE) inhibitors; IL-1 inhibitors (Interleukin-1-converting enzyme inhibitors, IL-1RA etc.) may be effective for the same reason. Other preferred combinations include Interleukin 11. Yet another preferred combination are other key players of the autoimmune response which may act parallel to, dependent on or in concert with TNFα inhibitors function; especially preferred are IL-18 antagonists including IL-18 antibodies or soluble IL-18 receptors, or IL-18 binding proteins. Yet another preferred combination are non-depleting anti-PSORIASIS 4 inhibitors. Yet other preferred combinations include antagonists of the co-stimulatory pathway CD 80 (B7.1) or CD 86 (B7.2) including antibodies, soluble receptors or antagonistic ligands.

In certain embodiments, agents which may be used in combination for the treatment of psoriasis which may be assessed according to the methods of the invention include one or more of TNFα inhibitors such as those described herein, methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g. IRAK, NIK, IKK, p38 or MAP kinase inhibitors), IL-1β converting enzyme inhibitors, TNFα converting enzyme (TACE) inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™ and p55TNFRIgG (Lenercept)), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol hcl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline hcl, sulfadiazine, oxycodone hcl/acetaminophen, olopatadine hcl, misoprostol, naproxen sodium, omeprazole, cyclophosphamide, rituximab, IL-1 TRAP, MRA, CTLA4-IG, IL-18 BP, anti-IL-18, Anti-IL15, BIRB-796, SCIO-469, VX-702, AMG-548, VX-740, Roflumilast, IC-485, CDC-801, and Mesopram.

In other embodiments, examples of therapeutic agents for psoriasis which may be assessed according to the methods of the invention alone or in combination with one or more therapeutic agents include the following: small molecule inhibitor of KDR (ABT-123), small molecule inhibitor of Tie-2, calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/nalact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, sulfasalazine.

In yet another embodiment, the methods of the invention may be used to determine or predict the long-term efficacy of a psoriasis treatment in combination with an antibiotic or antiinfective agent. Antiinfective agents include those agents known in the art to treat viral, fungal, parasitic or bacterial infections. The term, “antibiotic,” as used herein, refers to a chemical substance that inhibits the growth of, or kills, microorganisms. Encompassed by this term are antibiotic produced by a microorganism, as well as synthetic antibiotics (e.g., analogs) known in the art. Antibiotics include, but are not limited to, clarithromycin (Biaxin®), ciprofloxacin (Cipro®), and metronidazole (Flagyl®).

The methods of the invention may also be used to predict the long-term efficacy of a combination of agents that have a therapeutic additive or synergistic effect on the treatment of psoriasis. The combination of agents used within the methods or pharmaceutical compositions described herein also may reduce a detrimental effect associated with at least one of the agents when administered alone or without the other agent(s) of the particular pharmaceutical composition. For example, the toxicity of side effects of one agent may be attenuated by another agent of the composition, thus allowing a higher dosage, improving patient compliance, and improving therapeutic outcome. The additive or synergistic effects, benefits, and advantages of the compositions apply to classes of therapeutic agents, either structural or functional classes, or to individual compounds themselves.

### Pharmaceutical Compositions

The long-term efficacy pharmaceutical compositions comprising one or more substances for treating psoriasis, and a pharmaceutically acceptable carrier may be predicted according to the methods of the invention. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the substance for treating psoriasis.

The efficacy of compositions predicted according to the methods of the invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, Robinson, ed., Dekker, Inc., New York, 1978.

In certain embodiments, the substance for treating psoriasis may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In certain embodiments, the mode of administration is parenteral (e.g., intravenous, subcutaneous, intraperitoneal, intramuscular). In one embodiment, the psoriasis treatment is an antibody or other TNFα inhibitor which is administered by intravenous infusion or injection. In another embodiment, the antibody or other TNFα inhibitor is administered by intramuscular or subcutaneous injection. In one embodiment, the TNFα antibodies and inhibitors used in the invention are delivered to a subject subcutaneously. In one embodiment, the subject administers the TNFα inhibitor, including, but not limited to, TNFα antibody, or antigen-binding portion thereof, to himself/herself. In another embodiment the compositions are in the form of injectable or infusible solutions, such as compositions similar to those used for passive immunization of humans with other psoriasis treatments Formulations for treating psoriasis which may be assessed using the methods of the invention include protein crystal formulations which include a combination of protein crystals encapsulated within a polymeric carrier to form coated particles. The coated particles of the protein crystal formulation may have a spherical morphology and be microspheres of up to 500 micro meters in diameter or they may have some other morphology and be microparticulates. The enhanced concentration of protein crystals allows the antibody of the invention to be delivered subcutaneously. In one embodiment, the substances are delivered via a protein delivery system, wherein one or more of a protein crystal formulation or composition, is administered to a subject with psoriasis. Compositions and methods of preparing stabilized formulations of whole antibody crystals or antibody fragment crystals are also described in WO 02/072636, which is incorporated by reference herein. In one embodiment, a formulation comprising the crystallized antibody fragments described in PCT/IB03/04502 and U.S. Appln. No. 20040033228, incorporated by reference herein, are used to treat rheumatoid arthritis using the treatment methods of the invention.

Supplementary active compounds can also be incorporated into the compositions. In certain embodiments, a substance for treating psoriasis for use in the methods of the invention is coformulated with and/or coadministered with one or more additional therapeutic agents. Such combination therapies may advantageously utilize lower dosages of the administered therapeutic agents, thus avoiding possible side effects, complications or low level of response by the patient associated with the various monotherapies.

The pharmaceutical compositions of the invention may include a “therapeutically effective amount” or a “prophylactically effective amount” of substance for treating psoriasis. A “therapeutically effective amount” refers to an amount effective, at dosages and for periods of time necessary, to achieve the desired therapeutic result. A therapeutically effective amount of the substance may vary according to factors such as the disease state, age, sex, and weight of the individual, and the substance to elicit a desired response in the individual. A therapeutically effective amount is also one in which any toxic or detrimental effects of the substance are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

### Dosing Regimens

The long-term efficacy of dosing regimens may also be predicted according to the methods of the invention. In one embodiment, the long-term efficacy of a dosing regimen is predicted in a population of subjects having moderate to severe psoriasis. In one embodiment, the invention provides a method for predicting the long-term efficacy of a dosing regimen in a population of patients who have a subtherapeutic response to a therapy, who have failed to respond to a therapy, or have lost responsiveness to a therapy.

For example, the methods of the invention may be used to predict the long-term efficacy of a psoriasis treatment wherein the pharmaceutical composition containing one or more active ingredients is administered daily, every other day, thrice weekly, weekly, biweekly or monthly. In one embodiment, biweekly dosing includes a dosing regimen wherein doses of a psoriasis treatment are administered to a subject every other week beginning at week 1. In one embodiment, biweekly dosing includes a dosing regimen where doses of a psoriasis treatment are administered to a subject every other week consecutively for a given time period, e.g., 4 weeks, 8 weeks, 16, weeks, 24 weeks, 26 weeks, 32 weeks, 36 weeks, 42 weeks, 48 weeks, 52 weeks, 56 weeks, etc.

In one embodiment, treatment of psoriasis is achieved using multiple variable dosing methods of treatment. In one embodiment, the multiple variable dosing regimen includes increasing or escalating the dose of the psoriasis treatment over time. In one embodiment, the multiple dosing regimen comprising administering an initial loading dose of a psoriasis treatment to the subject at week 0. In one embodiment, the initial dose is given in its entirety on one day or is divided over 2 days. Following administration of the initial loading dose, a second dose, i.e., maintenance or treatment dose, of the psoriasis treatment may be administered to the subject. In one embodiment, the second dose is administered to the subject about one week after the first dose. Subsequent doses may be administered following the second dose in order to achieve treatment of the subject. Examples of such multiple variable dosing regimens are described in the Examples herein, and in PCT appln. no. PCT/US05/12007, incorporated by reference herein.

Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

The methods of the invention may be further be used to predict the efficacy of dosage regimens described herein in order to adjust the regimen to provide the optimum desired response, e.g., maintaining remission of psoriasis, in consideration of the teachings herein. It is to be noted that dosage values may vary with the type and severity of psoriasis. It is to be further understood that for any particular subject, specific dosage regimens may be adjusted over time according to the teachings of the specification and the individual need and the professional judgment of the person administering or supervising the administration of the compositions, and that dosage amounts and ranges set forth herein are exemplary only and are not intended to limit the scope or practice of the claimed invention.

### IV. Long-Term Efficacy Prediction

The invention provides a method for determining or predicting the long-term efficacy of a psoriasis treatment using population pharmacokinetic (PK) and pharmacodynamic (PD) modeling. The method may be used to predict the most appropriate dose and/or dosing interval of an agent or combination of agents, as well as whether and how to adjust doses for special populations (elderly, pediatric, patients with subtherapeutic responses to other agents). In addition, the method of the invention can be used to simulate a variety of clinical applications (e.g., treatment of different populations, different algorithms for adjusting doses and evaluating patient responses), in order to evaluate clinical trial designs (clinical trial simulation) or clinical practice.

To predict the long-term efficacy of a treatment for psoriasis, the method of the invention includes, in one embodiment, a pharmacokinetic model describing the pharmacokinetic profile of the agent or combination of agents used to treat the psoriasis. In one embodiment, the method of the invention comprises using of a one-compartment pharmacokinetic model. In another embodiment, the method of the invention comprises the use a one-compartment model with first-order absorption from a dose depot compartment. In another embodiment, the method of the invention comprises using a one-compartment model with first-order absorption from a dose depot compartment and first-order elimination from the central compartment. In another embodiment, the method of the invention comprises scaling the amount of drug in the central compartment by the apparent volume of distribution (V/F).

In another embodiment, the methods of the invention for predicting long-term efficacy of a psoriasis treatment include the use of a pharmacodynamic model and calculating one or more indices of psoriasis, e.g., PASI, PGA, DLQI, status. In preferred embodiment, the pharmacodynamic model is used to calculate the PASI score. In one embodiment, the pharmacodynamic model used in the methods of the invention an indirect response. In one embodiment, the pharmacodynamic model is a two-step indirect response model with an Eₘₐₓ concentration-response relationship. In a preferred embodiment, the pharmacodynamic model is a two-step indirect model with a linear concentration-response relationship. In another embodiment, the pharmacodynamic model used in the methods of the invention includes a residual error model. In one embodiment, additive and proportional error are used as a weighting factor. In another embodiment, the pharmacodynamic model used in the methods of the invention includes exponential inter-individual error terms (e.g., Kᵢₙ and K₄₀).

A pharmacokinetic model for an agent or combination of agents may be created according to standard models for pharmacokinetic data analysis which consist of a series of linear differential equations describe the mass transfer of drug from and to one or more “compartments”. Compartments in a pharmacokinetic model are hypothetical volumes that contain drug, and the differential equations describe the quantity (mass) of drug in the compartment as a function of time. The pharmacokinetic parameters (e.g., absorption rate constant, apparent clearance, apparent volume of distribution) for an agent or combination of agents to be used in these equations may be determined de novo following any number of standard techniques, or obtained from public or existing sources where available. For example, the concentration at a particular time point may be determined empirically by collecting a sample of a representative tissue (usually blood or plasma) and assaying that sample for the drug.

A model is then used to predict the concentration in the compartment by dividing the quantity of drug by the volume of distribution of the compartment. The volume of distribution of the compartment is a parameter estimated by fitting a model to observed data, using non-linear regression. The compartments used in these models may or may not correspond to any physiologic tissue. The “central compartment” describes the volume from which a sample is collected. This central compartment may correspond to the blood volume, or may be larger and correspond to the blood and tissues that equilibrate rapidly with the blood (i.e., mass transfer rate constants are large). The central compartment and any peripheral compartments are defined by the equations that describe the time course of the concentration of drug, not by any physiologic properties.

Pharmacodynamics refers to the study of fundamental or molecular interactions between drug and body constituents, which through a subsequent series of events results in a pharmacological response. For most drugs the magnitude of a pharmacological effect depends on time-dependent concentration of drug at the site of action. Pharmacodynamic modeling is approached in a similar fashion to pharmacokinetic modeling. A model is created that describes a given set of observed data. These observed data will include measurements such as PASI, PGA, DLQI or other quantity that are affected by the administration of drugs. In one embodiment, a model consistent with current understanding of the physiology of the drug is sought.

Methods for determining pharmacokinetic and pharmacodynamic models enumerated in current software (e.g., NONMEM, WinNonMix). NONMEM for example has 12 libraries of pharmacokinetic models. These include one compartment, one compartment with first order absorption, two compartment, two compartment with first order absorption, three compartment, three compartment with first order absorption, a general linear model (1-10 compartments) and a general nonlinear (1-10 compartments) and Michaelis-Menten kinetics. Other examples of software includes WinNonMix (Pharsight Corporation), Kinetica 2000 Population (Innaphase Corporation), and a procedure in SAS (SAS Institute) called NLMIXED. Various methods for creating pharmacokinetic models for drugs are described in U.S. Pat. Nos. 7,085,690, 6,542,858 and 7,043,415.

Patient populations that may be used in the methods of the invention are generally selected based on common characteristics. In one embodiment, the patient population contains subjects diagnosed with moderate to severe psoriasis who have not received a previous treatment for at least a period of time (e.g., one month, two months or more). In one embodiment, the patient population contains subjects diagnosed with moderate to severe psoriasis who have received treatment. In another embodiment, the patient population contains subjects diagnosed with psoriasis who are in remission as a result of receiving treatment. Such a patient population would be appropriate for predicting the long-term efficacy of as psoriasis therapy for maintaining remission in psoriasis in the given patient population. In another embodiment, the patient population has a common physical characteristic (e.g., age, gender, ethnicity, weight). In a related embodiment, the patient population is an adult population, e.g., older than 17 years of age or older than 18 years of age. In another embodiment, the patient population comprises subjects who have had a subtherapeutic response to a therapy, who has failed to respond to a therapy, or has lost responsiveness to a therapy.

Additional aspects of the invention pertain to a method of building a database, and computer program products useful for carrying out the methods of the invention. The method of building the database can comprise: receiving, in a computer system, pharmacokinetic and pharmacodynamic data for one or more psoriasis treatments from a plurality of subjects having psoriasis; and storing the data from each subject such that the data is associated with an identifier of the subject, such as a name of the subject, a physical characteristic or a numerical identifier coded to the identity of the subject.

Additional aspects of the invention pertain to a method of selecting a psoriasis treatment and/or dosing regimen for a subject using a database, and computer program products useful for carrying out the method. The method of selecting the psoriasis treatment and/or dosage regimen can comprise: identifying, in a database comprising a plurality of psoriasis subjects with similar physical characteristics or disease histories, a treatment regimen that has been predicted or confirmed to be effective in treating subjects with similar physical characteristics and/or disease histories.

Accordingly, as will be appreciated by one of skill in the art, the present invention may be embodied as methods, computer systems and/or computer program products. Thus, the invention may take the form of a hardware embodiment, a software embodiment running on hardware, or a combination thereof. Also, the invention may be embodied as a computer program product on a computer-usable storage medium having computer-usable program coded embodied in the medium. Any suitable computer readable medium may be utilized including disks, CD-ROMs, optical storage devices, magnetic storage devices, and the like.

For example, the methods or algorithms described in connection with the embodiments disclosed herein may be embodied directly in hardware, in a software module executable by a processor, or in a combination of both, in the form of control logic, programming instructions, or other directions, and may be contained in a single device or distributed across multiple devices. A software module may reside in RAM memory, flash memory, ROM memory, EPROM memory, EEPROM memory, registers, hard disk, a removable disk, a CD-ROM, direct access storage device (DASD), or any other form of storage medium known in the art. A storage medium may be coupled to the processor such that the processor can read information from, and write information to, the storage medium. In the alternative, the storage medium may be integral to the processor.

Computer program code for carrying out operations of the invention may be written in Visual Basic, (Microsoft Corporation, Redmond Wash.) and the like. However, the embodiments of the invention do not depend upon the use of a particular programming language. The program code may be executed on one or more servers or computers.

Computer system according to the invention suitably comprises a processor, main memory, a memory controller, an auxiliary storage interface, and a terminal interface, all of which are interconnected via a system bus. Note that various modifications, additions, or deletions may be made to the computer system within the scope of the present invention such as the addition of cache memory or other peripheral devices.

The processor performs computation and control functions of the computer system, and comprises a suitable central processing unit (CPU). The processor may comprise a single integrated circuit, such as a microprocessor, or may comprise any suitable number of integrated circuit devices and/or circuit boards working in cooperation to accomplish the functions of a processor. The processor suitably executes the PK/PD modeling computer programs of the present invention within its main memory.

The auxiliary storage interface allows the computer system to store and retrieve information from auxiliary storage devices, such as magnetic disk (e.g., hard disks or floppy diskettes) or optical storage devices (e.g., CD-ROM). One suitable storage device is a direct access storage device (DASD). A DASD may be a floppy disk drive which may read programs and data from a floppy disk. It is important to note that while the present invention has been (and will continue to be) described in the context of a fully functional computer system, those skilled in the art will appreciate that the mechanisms of the present invention are capable of being distributed as a program product in a variety of forms, and that the present invention applies equally regardless of the particular type of signal bearing media to actually carry out the distribution. Examples of signal bearing media include: recordable type media such as floppy disks and CD ROMS, and transmission type media such as digital and analog communication links, including wireless communication links.

The computer systems of the present invention may also comprise a memory controller, through use of a separate processor, which is responsible for moving requested information from the main memory and/or through the auxiliary storage interface to the main processor. While for the purposes of explanation, the memory controller is described as a separate entity, those skilled in the art understand that, in practice, portions of the function provided by the memory controller may actually reside in the circuitry associated with the main processor, main memory, and/or the auxiliary storage interface.

Furthermore, the computer systems of the present invention may comprise a terminal interface that allows system administrators and computer programmers to communicate with the computer system, normally through programmable workstations. It should be understood that the present invention applies equally to computer systems having multiple processors and multiple system buses. Similarly, although the system bus of the preferred embodiment is a typical hardwired, multidrop bus, any connection means that supports bidirectional communication in a computer-related environment could be used.

The main memory of the computer systems of the present invention suitably contains one or more computer programs relating to the PK/PD modeling of psoriasis treatment administration and an operating system. Computer program in memory is used in its broadest sense, and includes any and all forms of computer programs, including source code, intermediate code, machine code, and any other representation of a computer program. The term “memory” as used herein refers to any storage location in the virtual memory space of the system. It should be understood that portions of the computer program and operating system may be loaded into an instruction cache for the main processor to execute, while other files may well be stored on magnetic or optical disk storage devices. In addition, it is to be understood that the main memory may comprise disparate memory locations.

The invention is described with reference to flowchart illustrations of methods, and mathematical equations that can be implemented by computer program instructions. Such instructions may be provided to a processor of a computer and may also be stored in computer readable memory that can direct a computer to function in a particular manner, such that the instructions stored in the computer-readable memory are an article of manufacture.

The present invention is further illustrated by the following examples which should not be construed as limiting in any way.

### Example 1

The following analysis used a modeling and simulation approach to predict the long-term efficacy of methotrexate (MTX) in the treatment of moderate-to-severe psoriasis and to compare the predicted results with observed adalimumab efficacy data from Study M04-716.

Study M04-716 was a 16-week, Phase III, active- and placebo-controlled trial in North America and the EU in which patients with moderate-to-severe chronic plaque psoriasis were randomized to receive placebo, MTX, or adalimumab. At Week 16, PASI 75 response rates for adalimumab- and MTX-treated patients were 79.6% and 35.5%, respectively. Adalimumab had reached a plateau effect by Week 16; however, the efficacy of MTX was still increasing. Using the MTX dosage and PASI response data from Study M04-716, a population exposure-efficacy response model was developed using a non-linear mixed-effects population modeling (NONMEM) approach. Clinical trial simulations were then conducted to predict the plateau effect of MTX after long-term treatment.

MTX exposure was described using a one-compartment model with pharmacokinetic parameter values taken from those published in the literature because blood samples for the measurement of MTX concentrations were not collected in Study M04-716. A two-step indirect response model was used to describe the time course of PASI response via MTX treatment and the delay between the time course of MTX concentrations and reductions in PASI.

Using this model, the outcomes of Study M04-716 over the first 16 weeks were accurately reproduced. Clinical trial simulations were then conducted to predict the plateau effect of MTX on PASI score if MTX-treated patients in Study M04-716 had continued weekly treatment at the last dosage they received (mean±SD: 18.4±5.6 mg/week) for another 36 weeks (52 weeks in total from the start of Study M04-716). The simulations predicted that with a longer duration of treatment through one year, the PASI 75 response rate from MTX monotherapy would have been 47.8%.

Through a modeling and simulation approach, the plateau PASI 75 response rate from MTX monotherapy was predicted to be 47.8%, which is lower than that observed with adalimumab treatment (79.6%).

### Objectives

In Study M04-716, the safety, tolerability, and clinical efficacy of adalimumab vs. methotrexate (MTX) and vs. placebo in the treatment of moderate to severe chronic plaque psoriasis were evaluated over a 16-week period. The primary efficacy endpoint was the proportion of subjects achieving at least a 75% reduction in the Psoriasis Area and Severity Index (PASI) score (i.e., ≧PASI 75 response) at Week 16 relative to Baseline (Week 0).

The objective of the current analysis was to use population pharmacokinetic (PK) and pharmacodynamic (PD) modeling and simulation approach to predict the effect of MTX on PASI scores over a longer period of time than that was evaluated in Study M04-716.

### Background Information of Study M04-716

Study M04-716 was a 16-week multicenter, double-blind, and double-dummy study. A total of 271 subjects participated in this study. Subjects were randomized approximately 2:2:1 to one of three treatment regimens (N=53 for placebo, N=110 for MTX and N=108 for adalimumab). Over 90% of the subjects completed the study, and for the MTX group, 94.5% (104/110) subjects completed the study.

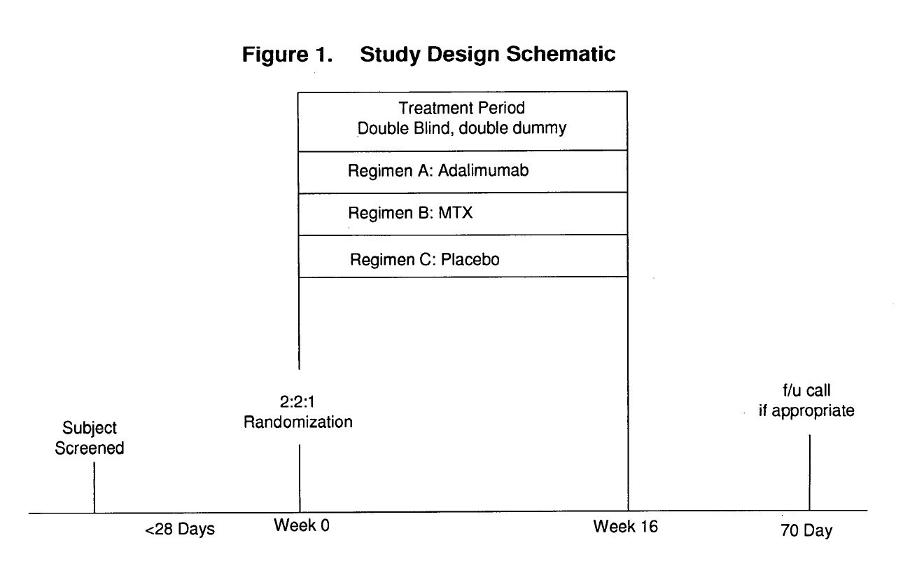

PASI scores were assessed prior to the first dose of study drug (Baseline) and at Weeks 1, 2, 4, 8, 12 and 16. Blood samples for the measurement of MTX concentrations were not collected during this study. The study design schematic is presented in FIG. 1.

Oral MTX was administered weekly in escalating doses from 7.5 to 25 mg. Dose escalation/titration was carried out according to the efficacy and safety criteria defined in the protocol. The summary statistics of actual MTX doses subjects received over time are shown in Table 1.

TABLE 1
MTX Dose (mg) Over Time

| | MTX | | |
|---|---|---|---|
| Visit | N | Mean ± SD | Median (Range) |
| Week 0 | 110 | 7.5 ± 0.00 | 7.5 (7.5-7.5) |
| Week 1 | 110 | 7.5 ± 0.00 | 7.5 (7.5-7.5) |
| Week 2 | 109 | 9.2 ± 2.96 | 10.0 (0.0-20.0) |
| Week 3 | 109 | 9.1 ± 3.15 | 10.0 (0.0-20.0) |
| Week 4 | 108 | 13.3 ± 4.57 | 15.0 (0.0-30.0) |
| Week 5 | 108 | 13.8 ± 3.36 | 15.0 (0.0-15.0) |
| Week 6 | 108 | 13.7 ± 3.67 | 15.0 (0.0-15.0) |
| Week 7 | 108 | 13.3 ± 4.14 | 15.0 (0.0-15.0) |
| Week 8 | 108 | 15.6 ± 5.53 | 15.0 (0.0-20.0) |
| Week 9 | 107 | 16.2 ± 5.31 | 15.0 (0.0-25.0) |
| Week 10 | 106 | 16.3 ± 5.25 | 15.0 (0.0-25.0) |
| Week 11 | 106 | 16.3 ± 4.95 | 15.0 (0.0-20.0) |
| Week 12 | 105 | 17.6 ± 6.69 | 20.0 (0.0-25.0) |
| Week 13 | 105 | 18.5 ± 5.90 | 20.0 (0.0-25.0) |
| Week 14 | 104 | 18.5 ± 5.89 | 20.0 (0.0-25.0) |
| Week 15 | 104 | 18.6 ± 5.80 | 20.0 (0.0-25.0) |

Missing data for any visit were imputed as 0 mg of MTX. However, MTX dropouts were not included in each visit analysis.

The primary efficacy endpoint, the PASI 75 response rate at Week 16, was statistically significantly higher in the adalimumab treatment group than the response rate in the placebo (79.6% vs. 18.9%; p<0.001) and MTX treatment groups (79.6% vs. 35.5%; p<0.001).

### Methods for Population Pharmacokinetic-Pharmacodynamic Modeling

### 1. Methods

The PK/PD model was built using a non-linear mixed-effects population modeling (NONMEM) approach with NONMEM software (double precision, Version VI) and a NMTRAN pre-processor. Models were compiled using the Intel Visual Fortran compiler (Version 9) on a dual processor workstation (DELL Precision 530) under the Windows 2000 (Service pack 4) operating system.

### 2. Description of Data

All 110 MTX-treated subjects in Study M04-716 were included in the population PK/PD analysis.

### Data for PK Modeling

The actual MTX doses and actual dosing times in Study M04-716 were used for the modeling. Because blood samples for the measurement of MTX were not collected in Study M04-716, the values of PK parameters (first-order absorption rate constant [Kₐ], apparent clearance [CL/F] and apparent volume of distribution [V/F]) from the literature were used.

### Data for PD Modeling

All the observed PASI scores over the 16-week period in MTX-treated subjects in Study M04-716 were used.

### 3. Population Pharmacokinetic-Pharmacodynamic Model Building

### Population Pharmacokinetic Model Building

A one-compartment model with first-order absorption from a dose depot compartment, and first-order elimination from the central compartment (shown below) was used to describe the PK profile of MTX.

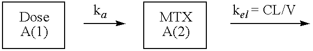

In the above schematic, A(1) and A(2) represent the amounts of MTX in the dose depot compartment and the central compartment, respectively. The amount in the central compartment was scaled by the apparent volume of distribution (V/F). Accordingly, C2(t)=A(2)(t)/(V/F) is the concentration of MTX in the central compartment at time t.

As mentioned above, because blood samples for the measurement of MTX were not collected in Study M04-716, the values of PK parameters (Kₐ, CL/F and V/F) from the literature were used in the PK/PD modeling (Table 2). All subjects were assumed to have the typical PK parameter values (i.e., intersubject and intrasubject variabilities were set to zero).

TABLE 2
Values of MTX Pharmacokinetic Parameters in the Literature and Used in NONMEM

| | | Values used in | | |
|---|---|---|---|---|
| | Values in the Literature | NONMEM | | Comments |
| CL/F | 12.4 L/h, 10.8 L/h, 11.5 L/h in patients with | 3 | L/day/kg | The average body weight |
| | psoriasis.¹ | | | in M04-716: approx. |
| | 2.1 mL/min/kg in patients with rheumatoid | | | 90 kg. |
| | arthritis (RA).² | | | |
| V/F | 0.55 L/kg in RA.² | 0.6 | L/kg | Patient population |
| | 0.4-0.8 L/kg (Vₛₛ).³ | | | unspecified for the Vₛₛ |
| | | | | value. |
| Kₐ | Tmax = 0.67-4 hrs in leukemic pediatric patients.³ | 10 | day⁻¹ | With CL/F = 3 L/day/kg, |
| | Tmax = 2 hrs in patients with psoriasis. | | | V/F day = 0.6 L/kg, and Kₐ = |
| | | | | 10 day⁻¹, the calculated |
| | | | | Tₘₐₓ would be 3.3 hrs, |
| | | | | within the range of |
| | | | | literature values. |

CL/F = apparent clearance, where F is the fraction of oral MTX dose reaching the systemic circulation.
V/F = apparent volume of distribution, where F is the fraction of oral MTX dose reaching the systemic circulation.
Vₛₛ = volume of distribution at steady state.
Kₐ = first-order absorption rate constant.

### Population Pharmacodynamic Model Building

PASI score was used to quantify the clinical response in population PD modeling. PASI is a continuous variable (range from 0 to 72) with higher scores reflecting more severe disease.

The anti-inflammatory effects of MTX occur at pharmacologically relevant concentrations of MTX.⁴ It has been reported that after MTX administration, MTX is taken up by cells via the reduced folate carrier and then is converted within the cells to polyglutamates. MTX polyglutamates are long-lived metabolites (persisting for weeks) that retain some of the antifolate activities of the parent compound.⁴ This can explain, at least partially, the increasing efficacy of MTX over the 16-week period of Study M04-716, even though MTX was only given once a week in the study and the half-life of MTX is only about 2 to 3 hours.² MTX having long-lived active metabolites can also explain the persistence of the clinical effect for several weeks even after the discontinuation of MTX doses.⁵

Several PD models were examined. The first model is an indirect response model with an inhibitory effect (Iₘₐₓ and IC₅₀) of Cₑ (concentration at an effect compartment) on Kᵢₙ. Kᵢₙ is the ‘synthesis rate’ into a compartment where PASI scores reside in. The 2^{nd} model examined was similar to the final PD model (see below), except that the rate into the 3^{rd} compartment is Kᵢₙ·(Eₘₐₓ/(1+EC₅₀/Cₚ)), rather than Kᵢₙ·Cₚ.

The final PD model is a two-step indirect model (as shown below). This model was found to be most appropriate to describe the delay hysteresis between the time course of MTX concentrations and clinical effect of PASI reduction, and the persistence of MTX clinical effect. In this model, Compartments 3 and 4 were added as delay/modulator compartments for triggering the observed PASI response.

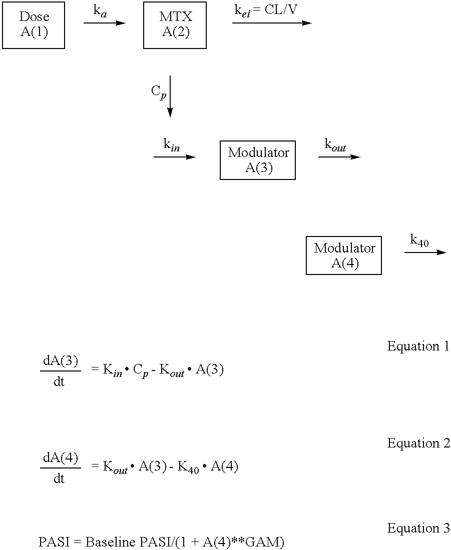

### Where:

Kᵢₙ and Kₒᵤₜ are the rate constants into and out of Compartment 3. The rate into Compartment 3 is regulated by MTX concentration at the central compartment (i.e., Cₚ). Kₒᵤₜ was set equal to Kᵢₙ.
K₄₀ is the rate constant out of Compartment 4, and it controls the persistence of PASI response.
PASI is the predicted PASI score, which equals to the baseline PASI score divided by a factor great than one, and the parameter ‘GAM’ influences the steepness of the functional relationship.

For the PD modeling, exponential error models were used to describe the inter-individual errors on the PD parameters Kᵢₙ and K₄₀.

Pᵢ=\hat{P}exp(ηᵢ^{p}) Equation 4

### Where:

Pᵢ is the true parameter value for individual i. It is assumed that Pᵢ follows a log-normal distribution;
\hat{P} is the typical value (population mean) of the parameter;
ηᵢ^{p} denotes the difference (in this case, the proportional difference) between the true value for individual i and the typical value for the population. The ηᵢ^{p} are independently, identically distributed with a mean of 0 and a variance of ω².

For the residual errors, additive and proportional (i.e., constant coefficient of variation) error models, as well as a combination of additive and proportional error models were tested.

Yᵢⱼ=PASIᵢⱼ+ε₁ᵢⱼ Equation 5

Yᵢⱼ=PASIᵢⱼ(1+ε₂ᵢⱼ) Equation 6

Yᵢⱼ=PASIᵢⱼ(1+ε₂ᵢⱼ)+ε₁ᵢⱼ Equation 7

### Where:

Yᵢⱼ is the jth observed PASI score in individual i;
PASIᵢⱼ is the jth model-predicted PASI score in individual i;
ε₁ᵢⱼ is the additive component of the residual intra-individual error for the jth measurement in individual i, with a mean of 0 and a variance of σ₂²;
ε₂ᵢⱼ is the proportional component of the residual intra-individual error for the jth measurement in individual i, with a mean of 0 and a variance of σ₁².

In addition, a different way of combining additive and proportional error as a weighting factor into the residual error model was examined.⁶

Yᵢⱼ=PASIᵢⱼ+w·ε₂ᵢⱼ Equation 8

w=(Theta(3)**2+PASIᵢⱼ*PASIᵢⱼ*Theta(4)**2)**0.5

or w=(1+PASIᵢⱼ*PASIᵢⱼ*Theta(5)**2)**0.5 Equation 9

### Where:

Theta(3) is the additive error standard deviation (SD);
Theta(4) is the proportional error coefficient of variation (CV);
Theta(5) is the ratio of the proportional error CV to the additive error SD.

When Equations 8 and 9 are used as the residual error model, the variance of ε₂ᵢⱼ (i.e., σ₂²) is fixed to one. Therefore, ε₂ᵢⱼ is assumed to following a normal distribution with a mean of 0 and a variance of 1 (i.e., N(0, 1)). When ε₂ᵢⱼ is multiplied by w, the residual error term (w·ε₂ᵢⱼ) is assumed to following a N(0, w²) distribution.

The evaluation criteria used to select an appropriate PK/PD model are described below:
1. When comparing hierarchical models, the objective function value (OFV) of a preferred model was significantly smaller than that of alternative model(s) based on the likelihood ratio test. The OFV is equal to −2 times the maximum logarithm of the likelihood of the data (−2LL). Non-hierarchical models are compared based on the AKAIKE criterion.
2. The observed and predicted PASI scores from a preferred model were more randomly distributed across the line of unity (a straight line with zero intercept and a slope of one) than those from alternative model(s).
3. Visual inspection of goodness-of-fit plots, parameter estimates and their standard errors, and changes in inter-subject and random residual errors indicated that the preferred model outperformed alternative model(s).

Because the objective of the population PK/PD analysis was not to identify significant covariates, covariate analyses for PD parameters were not performed.

The first-order conditional estimation (FOCE) with INTERACTION method was employed within NONMEM, and a diagonal structure of the n matrix was assumed.

The final population PK/PD model was validated using a bootstrap method (random resampling with replacement). The population estimates obtained from the final model were compared to the median, 2.5% and 97.5% percentiles of 1000 bootstrap replicates (2.5% and 97.5% percentiles are equivalent to the 95% confidence interval [CI]).

### Results

### 1. Population PD Modeling

The effect of MTX on PASI scores was modeled as an indirect response model with an inhibitory effect (Iₘₐₓ and IC₅₀) of Cₑ (concentration at an effect compartment) on the ‘synthesis rate’ into a compartment where PASI scores reside in (run1 to run2), a two-step indirect response model with a linear concentration-response relationship (run3 to run20), or a two-step indirect response model with an Eₘₐₓ concentration-response relationship (run30). The two-step indirect response model with a linear concentration-response relationship was found to be the most appropriate. Combining additive and proportional error as a weighting factor into the residual error model (run 8, OFV=2388.380) was found to be more appropriate than an additive (run3, OFV=2539.570) or proportional (run4, OFV=2539.413) error model alone or a simple combination of additive and proportional error model (run5, OFV=2419.583).

Therefore, a two-step indirect response model (with a linear concentration-response relationship), exponential inter-individual error terms on Kᵢₙ and K₄₀, and combining additive and proportional error as a weighting factor into the residual error model (run18) was identified as the final structural PD model. No covariates were analyzed.

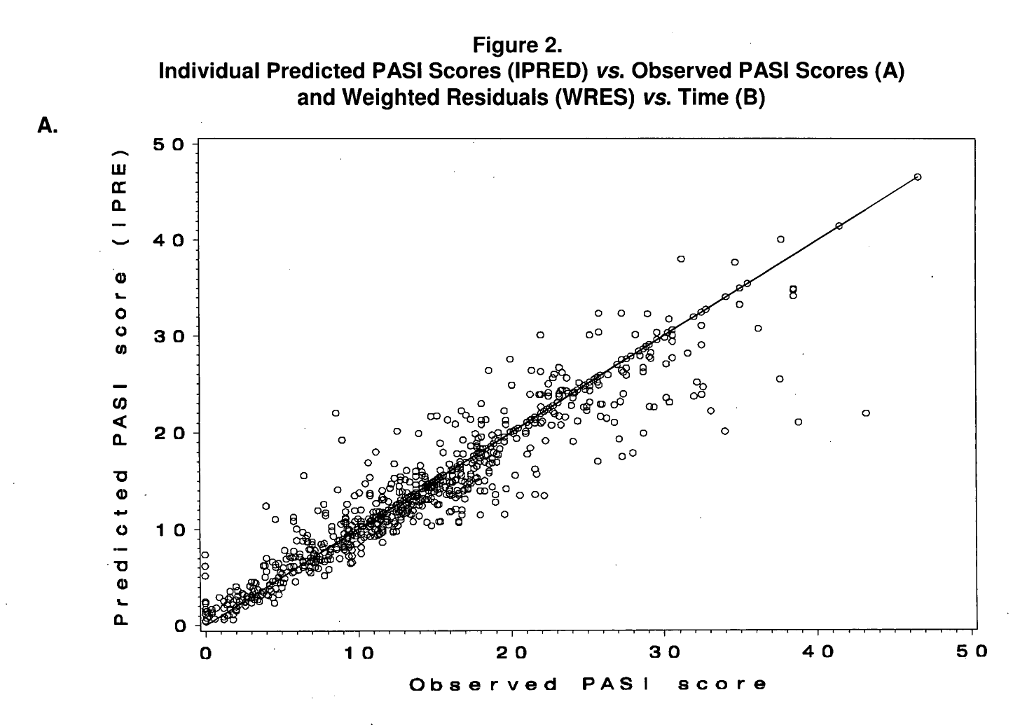

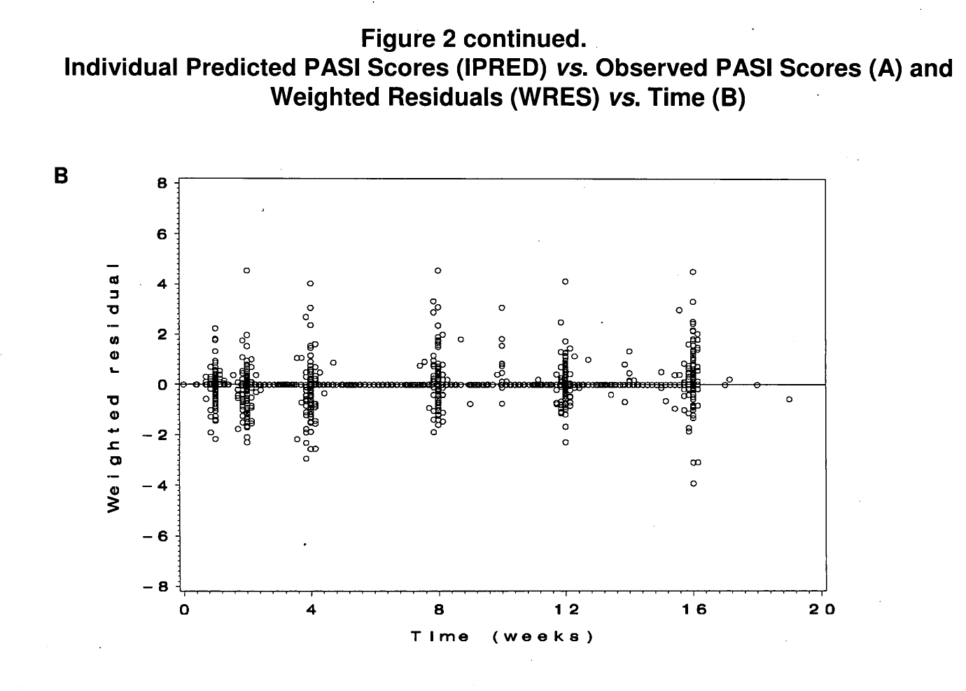

Goodness-of-fit plots for the final PD model are presented in FIG. 2. Generally, the final PD model adequately described the observed PASI scores in psoriasis subjects treated with MTX. The individual predicted vs. observed PASI scores were scattered around the line of unity, and the weighted residuals showed no major trends when plotted against time. These results indicated that the model was unbiased.

In addition, the final PD model was validated using a bootstrap method (random resampling with replacement). Among the 1000 bootstrap replicates, 881 replicates had successful minimization. The population estimates obtained from the final PD model were comparable to the medians and 95% confidence intervals of the corresponding estimates from the 881 bootstrap replicates with successful minimization. These results indicate that the final model was unbiased and stable, and demonstrate the usefulness of the exposure/clinical response model for simulation purposes.

Table 3 displays the PD parameter estimates from the final model (Run 18).

TABLE 3
Pharmacodynamic Parameter Estimates for the Final Model

| Parameter (unit) | Estimate (% RSE) | |
|---|---|---|
| Structural model parameters | | |
| Kᵢₙ (1/day) = THETA (1) | 3.83 | (15.0) |
| K₄₀ (1/day) = THETA (2) | 0.0203 | (35.1) |
| GAM = THETA (3) | 1.45 | (8.1) |
| Inter-individual variability parameters | | |
| % CV^{a} for Kᵢₙ | 69.1 | (21.0^{b}) |
| % CV^{a} for K₄₀ | 174.9 | (28.4^{b}) |
| Residual error parameters | | |
| w = (1 + E * E * THETA(4)**2)**0.5 | | |
| Y = E + w * EPS(1) | | |
| THETA (4) | 0.208 | (10.0) |

| | |
|---|---|
| σ² for EPS (1) | 1 Fixed |

^{a}For exponential inter-individual error model, ω*100 is an approximation of the % CV.
^{b}% RSE for ω² (variance for intersubject error).
% RSE (percent relative standard error of the estimate) = 100 * SE/Parameter Estimate

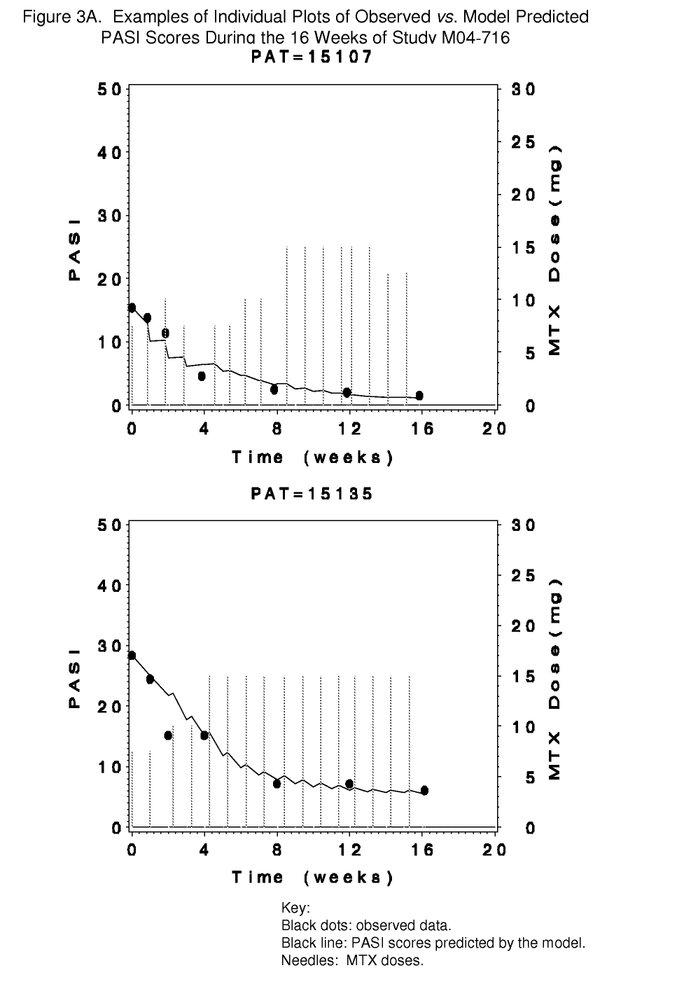

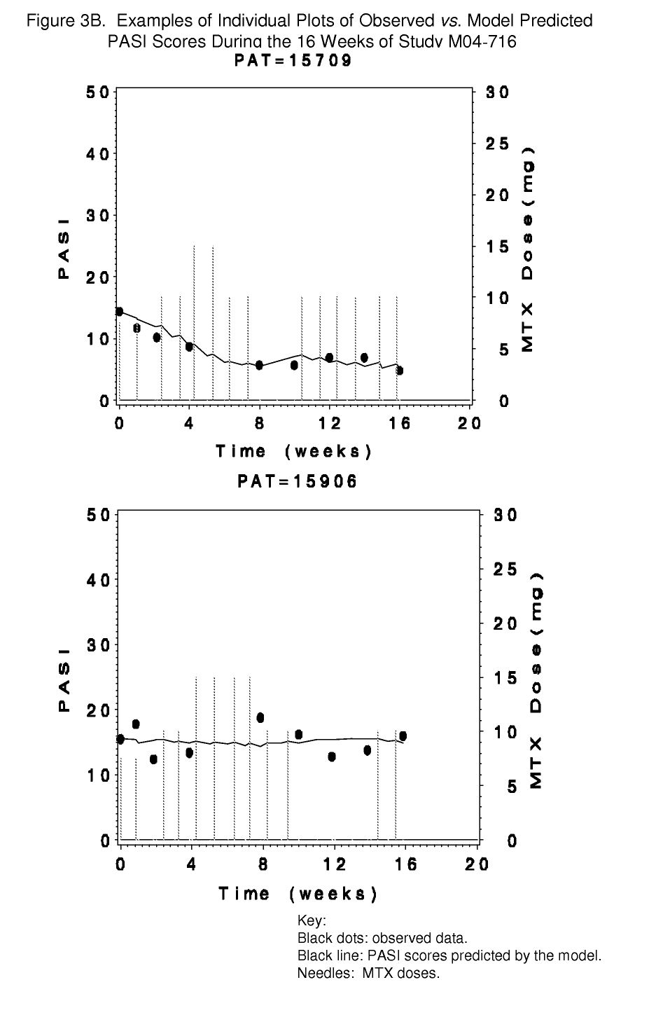

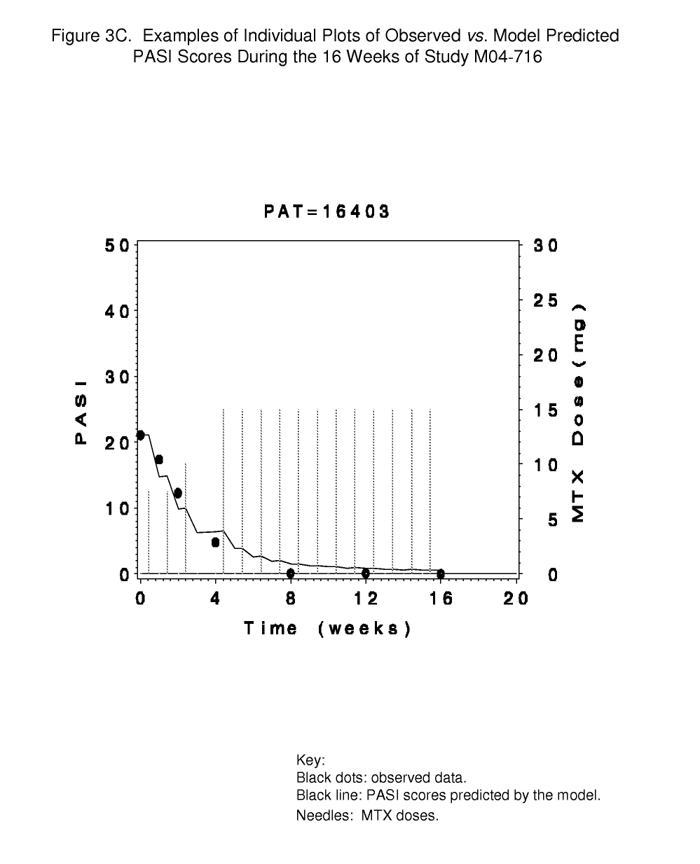

FIG. 3 shows examples of individual PASI score vs. time profiles (observed and predicted values), along with MTX doses.

2. Simulation of Long-Term MTX Treatment in Subjects with Psoriasis

Simulations were carried out using the final population PK/PD model to predict the plateau effect of MTX on PASI score if the subjects from Study M04-716 continued the weekly MTX dosing using the last MTX dose they received in Study M04-716 for another 36 weeks (i.e., 52 weeks in total from the start of Study M04-716).

Simulations were conducted using NONMEM software. The model structure and population PK/PD parameter estimates (Table 3), including intersubject and intrasubject variabilities, were used for the simulation. A total of 200 replicates (i.e., clinical trials) was run with 110 subjects in each replicate.

Same dataset as the one for the modeling was used, except that the last MTX dose for each subject in Study M04-716 was repeated weekly for another 36 weeks. Subject compliance from Week 16 through Week 52 was assumed to be 100%.

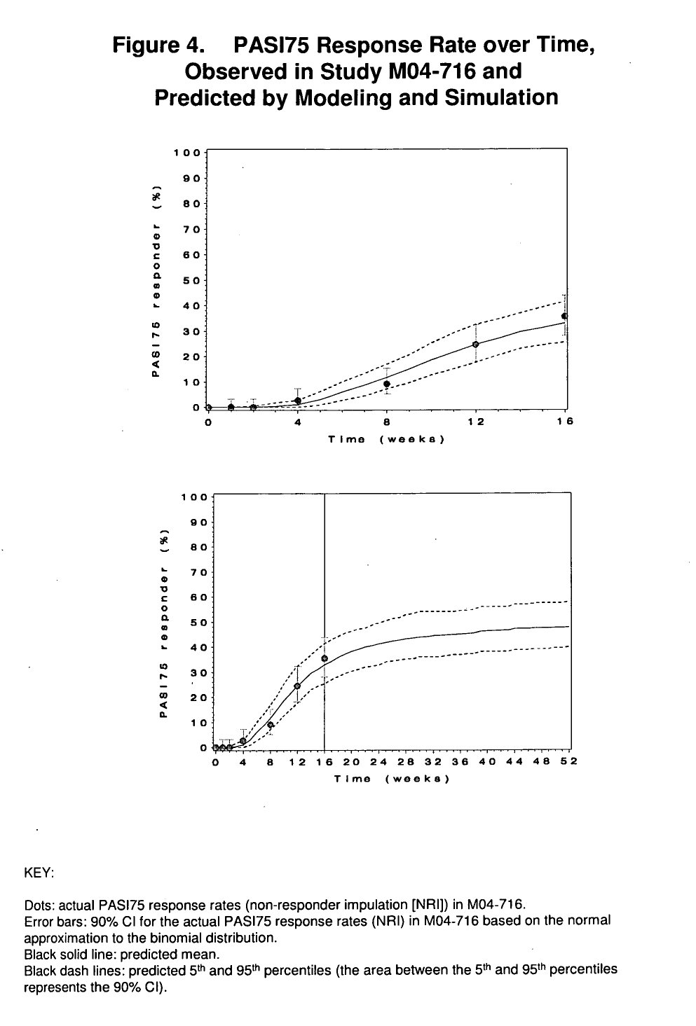

FIG. 4 and Table 4 show the PASI75 response rate over time, observed in Study M04-716 and predicted by modeling and simulation. The upper and lower panels of FIG. 4 show the profiles over the 16-week and 52-week periods, respectively.

TABLE 4
PASI75 Response Rate, Observed in Study M04-716
and Predicted by Modeling and Simulation

| | PASI75 Response Rate (90% CI*) | |
|---|---|---|
| Week | Actual (NRI) | Predicted |
| 1 | 0.0 (0.1, 3.2) | 0.0 (0.0, 0.0) |
| 2 | 0.0 (0.1, 3.2) | 0.0 (0.0, 0.4) |
| 4 | 2.7 (0.8, 7.2) | 1.1 (0.0, 2.7) |
| 8 | 9.1 (5.2, 15.2) | 11.8 (7.1, 17.0) |
| 12 | 24.5 (18.0, 32.3) | 24.6 (17.6, 32.4) |
| 16 | 35.5 (28.0, 43.7) | 32.9 (25.4, 41.4) |
| 24 | — | 41.3 (32.8, 49.6) |
| 52 | — | 47.8 (39.9, 57.9) |

*90% CI values for the actual PASI75 response rates (NRI) in M04-716 were based on the normal approximation to the binomial distribution.
90% CI values for the predicted PASI75 response rates were the values for the 5^{th} and 95^{th} percentiles.
NRI = non-responder impulation.

As shown in FIG. 4, the predicted PASI75 response rates were very similar to those observed in Study M04-716, indicating that the model is appropriate. As shown by the simulation, if weekly dosing of MTX was continued in the subjects from Study M04-716 using the last MTX dose they received, the PASI 75 response rate would be 47.8% at Week 52. Therefore, even with a longer duration of treatment, MTX response rates are predicted to be lower than those obtained with adalimumab treatment.

### Discussion and Conclusion

Using the MTX dose and PASI response data from the 16-week Study M04-716, a population PK/PD model was developed to describe the PASI response in subjects with psoriasis.

The final model included a PK component and a PD component. MTX PK were described using a one-compartment model with the values for Kₐ, CL/F and V/F fixed to those published in the literature since blood samples for the measurement of MTX concentrations were not collected in the study.

The PD component was described using a two-step indirect response model (with a linear concentration-response relationship), exponential inter-individual error terms on Kᵢₙ and K₄₀, and combining additive and proportional error as a weighting factor into the residual error model.

The final PK/PD model was found to be appropriate and unbiased. The model accurately reproduced the outcome of Study M04-716 over the first 16 weeks.

Utilizing this PK/PD model, clinical trial simulations were conducted to predict the plateau effect of MTX on PASI score if the subjects in Study M04-716 continue the weekly MTX dosing using the last MTX dose they received in Study M04-716 for another 36 weeks (i.e., 52 weeks in total from the start of Study M04-716).

The simulation results show that if we continue weekly dosing the subjects in Study M04-716 using the last MTX dose they received, the PASI 75 response rate would be 47.8% at Week 52. Therefore, even with a longer duration of treatment, MTX response rates never reached those obtained by adalimumab treatment.

### LIST OF ABBREVIATIONS AND DEFINITIONS

CL/F Apparent clearance
CV Coefficient of variation
df Degrees of freedom
ETA Inter-individual random effect
Kₐ Absorption rate constant
NONMEM Non-Linear Mixed-Effects Modeling
OFV Objective function value
PD Pharmacodynamic
PK Pharmacokinetic
P5 5^{th} percentile
P95 95^{th} percentile
PASI Psoriasis Area and Severity Index
SD or Std Standard deviation
V/F Apparent volume of distribution
WT Body weight

### REFERENCES

1. Chladek J. et al. Pharmacokinetics of low doses of methotrexate in patients with psoriasis over the early period of treatment. Eur J Clin Pharmacol 1998, 53: 437-444
2. Goodman & Gilman. In: Hardman J G, Limbird L E, Molinoff P B, Ruddon R W, Gilman A G, editors. The Pharmacological Basis of Therapeutics, 9th Edition. New York: McGraw Hill, 1996.
3. Methotrexate Sodium Tablets (Rheumatrex®) product label, 2003
4. Chan E S L, Cronstein B N. Molecular action of methotrexate in inflammatory diseases. Arthritis Res 2002, 4:266-273.
5. Van Dooren-Greebe R J, et al. Interruption of long-term methotrexate treatment in psoriasis. Act Derm Venereol 1995, 75: 393-396.
6. Course Material Intermediate Level Workshop in Population Pharmacokinetic Data Analysis using the NonMem System, 16-17 Oct., 2003, Uppsala, University of California San Francisco, Lecture 2 Model-Building Graphics, Page 3.

### Example 2

The goal of this study was as follows: a modeling and simulation approach was used to predict the long-term efficacy of methotrexate (MTX) in the treatment of moderate to severe psoriasis and to compare the predicted results with observed adalimumab efficacy data from the Phase III Comparative Study of HUMIRA vs. Methotrexate vs. Placebo In Ps0riasis Patients (CHAMPION) study.

The methods used in this study include the following: CHAMPION was a 16-week, Phase III, active- and placebo-controlled trial in North America and the European Union (EU) in which patients with moderate to severe chronic plaque psoriasis were randomized to receive placebo (N=53), MTX (N=110), or adalimumab (N=108). At Week 16, Psoriasis Area and Severity Index (PASI) 75 response rates for adalimumab- and MTX-treated patients were 79.6% and 35.5%, respectively. Adalimumab had reached a plateau effect by Week 16; however, the PASI 75 response rate for MTX was continuing to increase. Using the MTX dosage and PASI response data from CHAMPION, a population exposure-efficacy response model was developed with a nonlinear mixed-effects population modeling (NONMEM) approach. Computer-aided clinical trial simulations were then conducted to predict the plateau effect of MTX after long-term treatment.

MTX exposure was described using a 1-compartment model. Because blood samples for the measurement of MTX concentrations were not collected in the CHAMPION trial, pharmacokinetic parameter values from the literature were used in the model. A 2-step indirect-response model was used to describe the time course of PASI response during MTX treatment and the delay between the time course of MTX concentrations and reductions (improvement) in PASI scores.

The results of this study are summarized as follows: Using this model, the outcomes of CHAMPION over the first 16 weeks were accurately reproduced. Clinical trial simulations were then conducted to predict the plateau effect of MTX on PASI score if MTX-treated patients in CHAMPION had continued weekly treatment at the last dosages received (mean±SD: 18.4±5.6 mg/wk) for another 36 weeks (52 weeks in total from the start of CHAMPION). The simulations predicted that, with a longer duration of treatment through 1 year, the PASI 75 response rate for MTX monotherapy would have reached 47.8%.

In conclusion, through a computer-modeling and simulation approach, the plateau of the PASI 75 response rate for MTX monotherapy was predicted to reach 47.8%, which was much lower than the rate observed with adalimumab treatment (79.6%).

### Introduction

Psoriasis is a chronic, inflammatory proliferative disease characterized by marked inflammation and thickening of the epidermis, resulting in thick, scaly plaques on the skin. Patients with moderate to severe psoriasis may require long-term, systemic treatment.

The cytokine tumor necrosis factor (TNF) is involved in the pathogenesis of psoriasis. TNF is specifically targeted by the fully human monoclonal antibody Adalimumab (ADA). In December 2007, adalimumab received EMEA approval for treatment of plaque psoriasis. ADA has also been approved for treating patients with rheumatoid arthritis, psoriatic arthritis, ankylosing spondylitis, juvenile idiopathic arthritis (in the United States), and Crohn's disease.

Methotrexate (MTX) is currently the most frequently prescribed systemic therapy for psoriasis in the European Union. Its use may be complicated by bone marrow suppression, gastrointestinal and hepatic toxicities, liver failure, and even death.

In CHAMPION, a 16-week, Phase III, active- and placebo-controlled trial in patients with psoriasis, adalimumab treatment resulted in a 79.6% Psoriasis Area and Severity Index (PASI) 75 response rate at Week 16, which was statistically significantly greater than the MTX response rate of 35.5% (p<0.001). Adalimumab had reached a plateau of efficacy by Week 16; however, the PASI 75 response rate for MTX was continuing to increase.

### Objective

The aim of the present invention was to predict the long-term efficacy of MTX in the treatment of moderate to severe psoriasis using a computer-modeling and simulation approach and to compare the predicted results with observed adalimumab efficacy data from the Phase III CHAMPION study.

### Methods

Subjects were selected based on the following criteria; psoriasis patients had moderate to severe plaque psoriasis (≧10% body surface area involvement and a PASI score of ≧10) at the baseline visit, patients displayed stable plaque psoriasis for at least 2 months prior to screening and patients had no previous exposure to TNF antagonists or MTX. In addition, the subjects were candidates for systemic therapy or phototherapy.

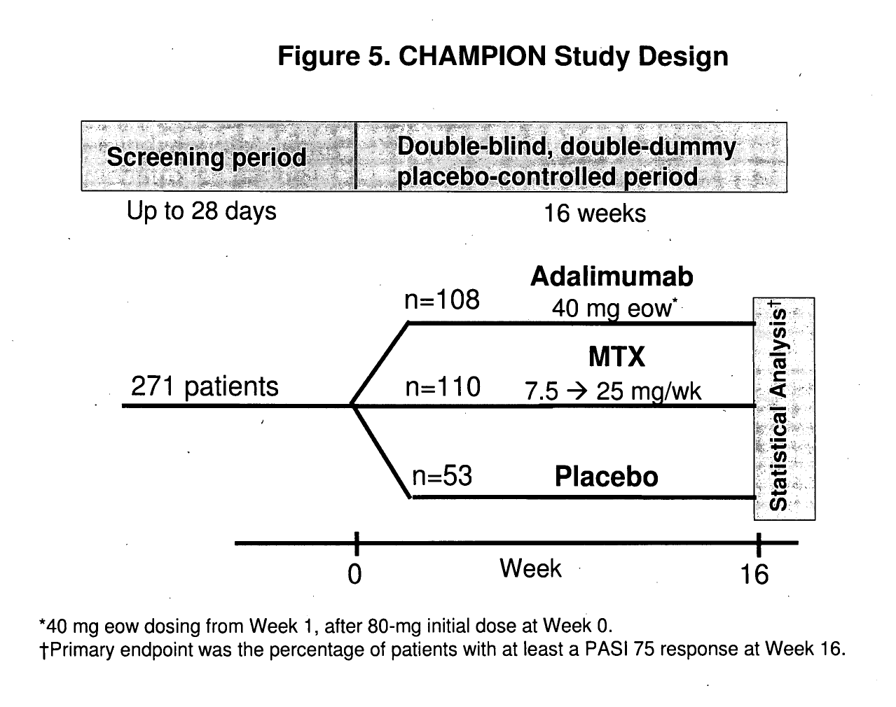

Selected patients were randomized in a 2:2:1 ratio to adalimumab, MTX, or placebo. The ADA treatment group received 40 mg every-other-week (eow) injections from Week 1, following an 80-mg initial dose. The MTX treatment group received oral MTX, given weekly in escalating doses from 7.5 to 25 mg. Dose escalation was carried out according to the efficacy and safety criteria defined in the study protocol. All study patients received injections as well as oral tablets, regardless of treatment group assignment (double-dummy design) (FIG. 5). Patient outcome was measured using a PASI 75 response: a 75% reduction (improvement) in PASI score at Week 16 compared with the baseline score.

Statistical analysis was performed using a Cochran-Mantel-Haenszel (CMH) test stratified by country was used to assess treatment differences. In addition, pharmacokinetic models were designed using nonlinear mixed-effects population modeling (NONMEM) software, and computer-aided clinical trial simulations were conducted to predict the maximum plateau effect of PASI 75 response rates achieved with long-term treatment with MTX.

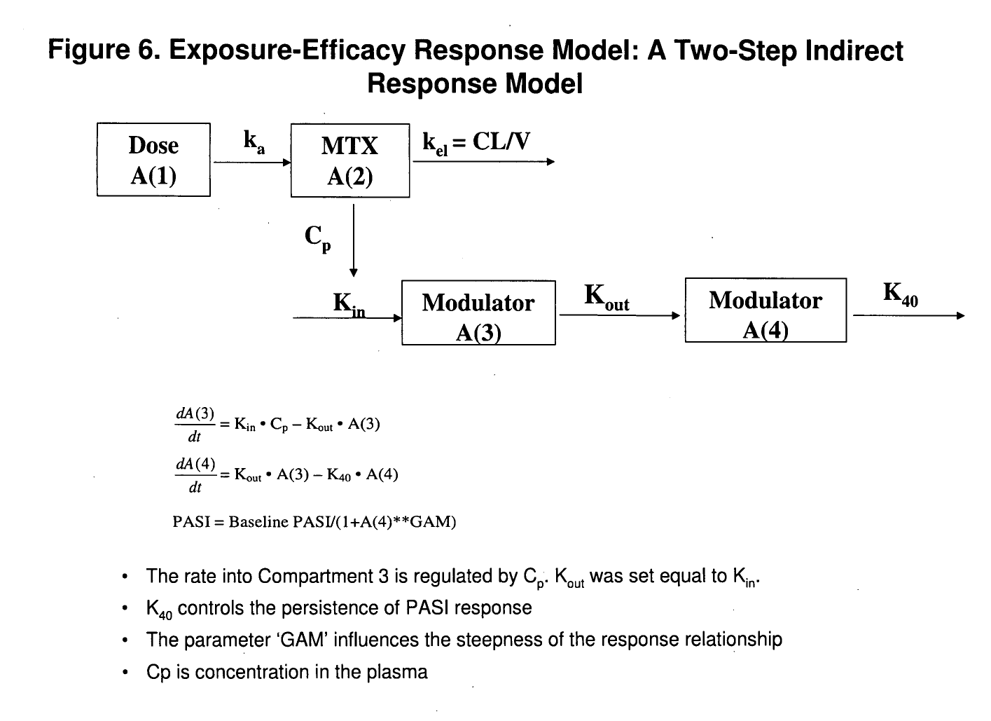

Population Exposure-Efficacy Response Modeling was performed by constructing a model using the 16-week data (MTX doses and PASI scores) in MTX-treated patients (FIG. 6). No blood samples for MTX concentration measurement were collected. Therefore, pharmacokinetic parameter values from the literature [1-3] were used.

Apparent clearance (CL/F)=3 L/day/kg

Apparent volume of distribution (V/F)=0.6 L/kg

First-order absorption rate constant (Ka)=10 day−1

A 1-compartment model was used to describe the concentration-time profile of MTX. A 2-step indirect response model was used to describe the effect of MTX on PASI score reduction.

### Results

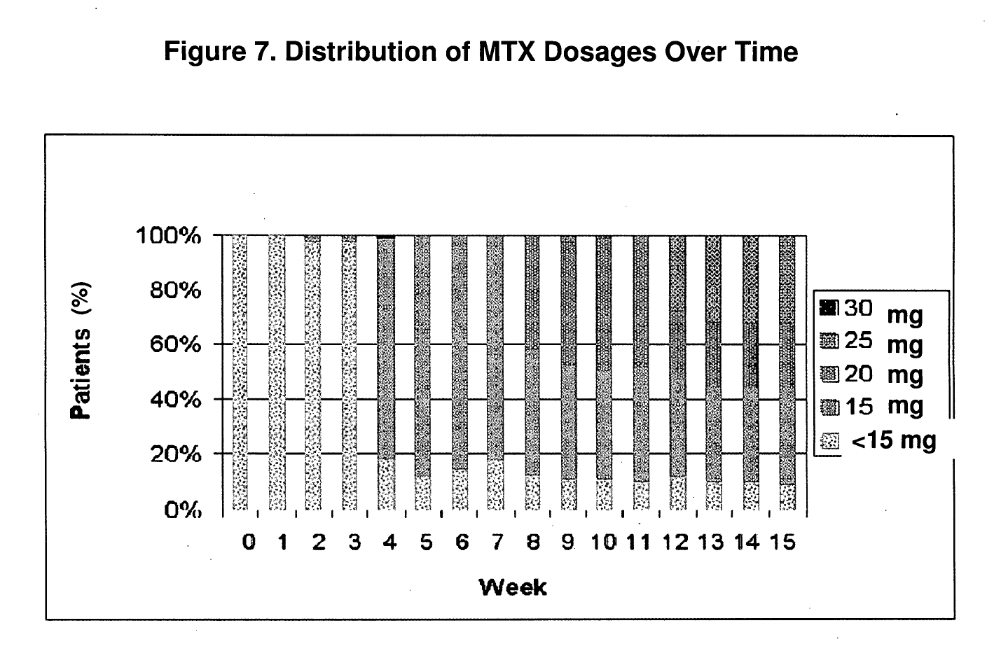

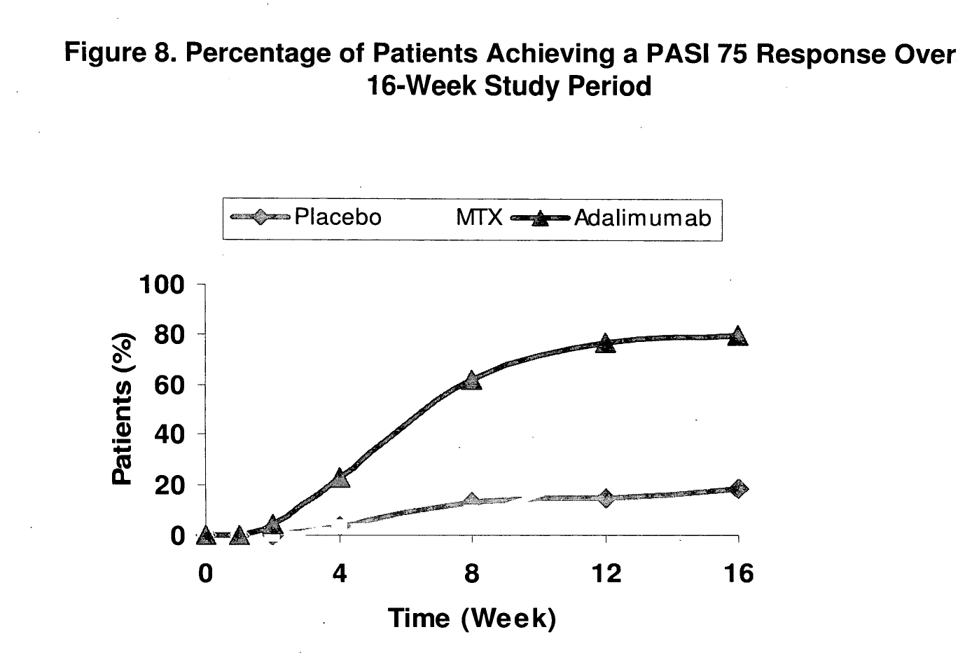

In the present study, a total of 271 patients were randomized and 256 (94%) completed the 16-week study. At baseline, disease severity (PASI score) and demographic characteristics were similar across treatment groups (Table 5). For the MTX treatment group, oral MTX was given weekly in escalating doses from 7.5 to 25 mg. Shown in FIG. 7 is the MTX dosage distribution over the study time course. Adalimumab treatment resulted in a statistically significantly greater PASI 75 response rate at Week 16 (79.6%) compared with the placebo (18.9%; p<0.001) and MTX treatment groups (35.5%; p<0.001). Adalimumab had reached a plateau effect by Week 16; however, the PASI 75 response rate for MTX was continuing to increase (FIG. 8). To predict the efficacy that would have been achieved had MTX therapy been continued for 1 year, a mathematical model was developed. To test the validity of the model, the results predicted for Weeks 0 to 16 were compared with those actually observed.

Goodness-of-fit plots demonstrate the adequacy of the fitting of the model to the data (FIG. 2). Individual predicted versus observed PASI scores were scattered around the line of unity and weighted residuals showed no major trends over time. FIG. 3 shows the ability of the model to predict individual patient responses. The blue line represents predicted responses and the red dots represent actual data.

Simulations were carried out to predict the plateau effect of MTX on PASI score if MTX-treated patients in CHAMPION had continued weekly treatment at the last dosage received for another 36 weeks (ie, 52 weeks in total). A total of 200 replicates (i.e., simulated clinical trials) were run with 110 patients in each replicate and the model accurately reproduced the outcomes of CHAMPION over the first 16 weeks (FIG. 4, top panel). The simulation results indicated that if weekly dosing of MTX had been continued in patients using the last MTX dosage received, the predicted PASI 75 response rate would have reached 47.8% at Week 52 (FIG. 4, bottom panel). The simulated PASI 75 response rate for MTX at Week 52 was approximately 12% greater than that at Week 16.

TABLE 5
Baseline Demographic and Clinical Characteristics
of Patients by Treatment Group

| | Placebo | Methotrexate | Adalimumab |
|---|---|---|---|
| | (n = 53) | (n = 110) | (n = 108) |
| Age, yrs* | 40.7 | 41.6 | 42.9 |
| Male, % | 66.0 | 66.4 | 64.8 |
| White, % | 92.5 | 95.5 | 95.4 |
| Ps duration, yrs* | 18.8 | 18.9 | 17.9 |
| Body weight, kg* | 82.6 | 83.1 | 81.7 |
| BSA, % affected* | 28.4 | 32.4 | 33.6 |
| PASI score* | 19.2 | 19.4 | 20.2 |
| PsA, % | 20.8 | 17.3 | 21.3 |

*Mean values.
Ps = psoriasis; PsA = psoriatic arthritis.

### CONCLUSIONS

Using the MTX dosage and PASI response data from the 16-week CHAMPION study, a population exposure-efficacy response model was developed. The model was successful in accurately reproducing the outcomes of CHAMPION over the first 16 weeks. Using this model, the plateau of the PASI 75 response rate for MTX monotherapy was predicted to reach 47.8% at Week 52. This is substantially lower than the actual rate observed at Week 16 with adalimumab treatment (79.6%).

### REFERENCES

1. Chladek J, et al. Eur J Clin Pharmacol. 1998; 53:437-444.
2. Goodman & Gilman. In: Hardman J G, Limbird L E, Molinoff P B, Ruddon R W, Gilman A G, eds. The Pharmacological Basis of Therapeutics, 9th Edition. New York: McGraw Hill, 1996:1758.
3. Methotrexate Sodium Tablets (Rheumatrex®) [package insert]. Cranbury, N.J.: STADA Pharmaceuticals, Inc.; 2003. Available at: http://www.rheumatrex.info/pdf/RheumatrexPackageInsert.pdf.

### EQUIVALENTS

The previous description of the disclosed embodiments is provided to enable any person skilled in the art to make or use the present invention. Those skilled in the art will recognize, or be able to ascertain using no more than routine experimentation, many equivalents to the specific embodiments of the invention described herein. Such equivalents are intended to be encompassed by the following claims. Various modifications to these embodiments will be readily apparent to those skilled in the art, and the generic principles defined herein may be applied to other embodiments without departing from the spirit of scope of the invention. Moreover, nothing disclosed herein is intended to be dedicated to the public regardless of whether such disclosure is explicitly recited in the claims. The contents of all references, patents and published patent applications cited throughout this application are incorporated herein by reference.

What is claimed:

1. A method for predicting the efficacy of a psoriasis treatment comprising,
providing a pharmacokinetic model describing the pharmacokinetic profile of the treatment;
providing a pharmacodynamic model of the compound; and
calculating a value for psoriasis index from the pharmacodynamic model, thereby predicting the efficacy of the psoriasis treatment.

2. The method of claim 1, wherein the pharmacokinetic model contains a central component, the central component describing the concentration of the compound at a given time.

3. The method of claim 2, wherein the pharmacokinetic model is a one-compartment model.

4. The method of claim 2, wherein the pharmacokinetic model is a one-compartment model with first-order absorption from a dose depot compartment.

5. The method of claim 2, wherein the pharmacokinetic model is a one compartment model with first-order absorption from a dose depot compartment and first-order elimination from a central compartment.

6. The method of claim 2, wherein the amount of a compound for treating psoriasis in the central compartment is scaled by apparent volume of distribution.

7. The method of claims 1, wherein the psoriasis index is a psoriasis area and severity index (PASI).

8. The method of claims 1, wherein the pharmacodynamic model is a two-step indirect model with a linear concentration-response relationship.

9. The method of claims 1, wherein additive and proportional errors are used as a weighting factor in the pharmacodynamic model.

10. The method of claim 9, further comprising exponential inter-individual error.

11. The method of claim 1, wherein the psoriasis treatment is a systemic treatment.

12. The method of claim 11, wherein the systemic treatment comprises a corticosteroid.

13. The method of claim 11, wherein the systemic treatment comprises a TNFα inhibitor.

14. The method of claim 11, wherein the psoriasis treatment is methotrexate.

15. The method of claim 1, wherein the psoriasis treatment comprises two agents for treating psoriasis.

16. The method of claim 1, wherein the psoriasis treatment comprises a weekly dosing regimen.

17. The method of claim 1, wherein the psoriasis treatment comprises a biweekly dosing regiment.

18. The method of claim 1, wherein the psoriasis treatment comprises a multiple variable dose regimen.

19. A method of claim 1, comprising predicting the efficacy of the psoriasis treatment for at least 6 months.

20. The method of claim 1, comprising predicting the efficacy of the psoriasis treatment for at least 12 months.

21. The method of claim 1, comprising predicting the efficacy of the psoriasis treatment in a population.

22. The method of claim 21, comprising predicting the efficacy of the psoriasis treatment in a subpopulation of individuals having a common characteristic selected from the group consisting of age, gender, race and non-responsiveness to a previous psoriasis treatment.

23. The method of claim 1, comprising predicting the efficacy of the psoriasis treatment for an individual.

24. A method of selecting a psoriasis treatment comprising:
predicting the efficacy of a first psoriasis treatment using pharmacokinetic and pharmacodynamic models to create a pharmacodynamic profile of the first psoriasis treatment;
predicting the efficacy of a second psoriasis treatment using pharmacokinetic and pharmacodynamic models to create a pharmacodynamic profile of the second psoriasis treatment;
comparing the pharmacodynamic profile of the first psoriasis treatment to the pharmacodynamic profile of the second psoriasis treatment; and
selecting the psoriasis treatment having the higher predicted efficacy.

25. The method of claim 24, wherein the first and second psoriasis treatments comprised different active compounds for treating psoriasis.

26. The method of claim 24, wherein the first and second psoriasis treatments comprise the same substance but different dose regiments.

27. The method of claim 24, wherein the first and second psoriasis treatments comprise different pharmaceutical formulations of the same active compound.

28. A method for predicting the efficacy of a compound for the treatment of psoriasis comprising:
creating a pharmacokinetic model describing the pharmacokinetic profile of the compound, wherein the pharmacokinetic model contains a central compartment, the central compartment describing a concentration of the compound at a given time;
creating a two-step pharmacodynamic model wherein a concentration regulates the rate of the compound into the second step of the model; and
calculating a psoriasis area and severity index from the pharmacodynamic model, thereby predicting efficacy of the compound for the treatment of psoriasis.

29. The method of claim 28, further comprising calculating the inter-individual errors for the rate into the second step of the pharmacodynamic model and the rate out of the second step of the pharmacodynamic model and/or creating a residual error model combining additive and proportional error as a weighting factor.

30. A computer program product for predicting the efficacy of a psoriasis treatment comprising:
a computer readable medium with a program stored on the medium describing a pharmacokinetic model and pharmacodynamic model for determining the pharmacokinetic and pharmacodynamic profiles of the psoriasis treatment;
executable instructions that when executed cause a processor to perform operations comprising: receiving, in a computer system, data from one or more individuals administered the psoriasis treatment; and applying the pharmacokinetic and pharmacokinetic models to thereby predict the efficacy of the psoriasis treatment.

31. A method of building a database for use in predicting the efficacy of a psoriasis treatment for an individual comprising:
a computer readable medium with a program stored on the medium describing a pharmacokinetic model and pharmacodynamic model for determining the pharmokinetic and pharmacodynamic profiles of the psoriasis treatment; and
a computer receiving, in a computer system, data from a plurality of subjects having received treatment for psoriasis; and storing the data such that physical characteristics, psoriasis treatment received, dose regimen and responsiveness for each subject is associated with an identifier.

32. A method of selecting a psoriasis treatment for a subject comprising:
identifying, in a database comprising data from a plurality of psoriasis subjects, the predicted efficacy of one or more psoriasis treatments determined from the pharmacokinetic and pharmacodynamic profiles calculated from data obtained from subjects having one or more characteristics in common the subject to be treated; and
selecting a psoriasis treatment for the subject based on the predicted efficacy of the treatment.
